(19) United States
(12) Patent Application Publication
(10) Pub. No.: US 2010/0273667 A1
(43) Pub. Date: Oct. 28, 2010

(54) Cell culture well-plates having inverted colloidal crystal scaffolds

(75) Inventors: Nicholas A. Kotov, Ypsilanti, MI (US); Jungwoo Lee, Ann Arbor, MI (US); Meghan J. Cuddihy, Ann Arbor, MI (US)

Correspondence Address: HARNESS, DICKEY & PIERCE, P.L.C., P.O. BOX 828, BLOOMFIELD HILLS, MI, 48303, US

(73) Assignee: THE REGENTS OF THE UNIVERSITY OF MICHIGAN

(21) Appl. No.: 11/656,362

(22) Filed: Jan. 22, 2007

Related U.S. Application Data
(60) Provisional application No. 60/772,283, filed on Feb. 10, 2006.

(51) Int. Cl. C12Q 1/02; C12N 5/071; C40B 30/04; C12Q 1/68; C08J 9/00; B01J 19/08; B29C 67/20

(52) U.S. Cl. 506/9; 435/396; 435/29; 435/6; 521/50.5; 422/135

(57) ABSTRACT

A three dimensional inverted colloidal crystal scaffold is described which comprises a substrate having at least one well. The scaffold also includes a three dimensional matrix comprising a transparent biocompatible polymeric network containing microspherical voids. The microspherical voids are each connected to at least one other void through inter-connecting pores. Additionally, an apparatus for producing such a colloidal crystal scaffold is described. Methods for making the inverted colloidal crystal scaffold, for using the scaffold and for identifying the effects of a drug, pharmaceutical or toxin on a living cell using the inverted colloidal crystal scaffold are also disclosed.

### CROSS-REFERENCE TO RELATED APPLICATIONS

This application claims the benefit of U.S. Provisional Application No. 60/772,283, filed on Feb. 10, 2006. The disclosure of the above application is incorporated herein by reference.

### GOVERNMENT RIGHTS

This disclosure was made with government support under DARPA Grant No. 049706. The Government has certain rights in the disclosure.

### FIELD

The present disclosure relates to cell culture and, more particularly, relates to microplates having a three-dimension matrix scaffold.

### BACKGROUND

The statements in this section merely provide background information related to the present disclosure and may not constitute prior art.

The majority of cell culture studies are performed on two-dimensional (2-D) surfaces that include well microplates, tissue culture plates, tissue culture flasks, and Petri-dishes. In particular, cell culture microplates, which contain a large number of small identical wells for example 2, 4, 8, 16, 24, 48, 96, 384, and up to 1536 wells are used widely because they are ideal for the study of low numbers of cells and high throughput cellular assays. These plates are a standard in analytical research and clinical diagnostic testing. The disadvantage of conventional cell culture in microplates and flasks lies in the limitation to 2-D culture.

The importance of a three-dimensional (3-D) cell culture substrate has been demonstrated in various cellular adhesion, migration, proliferation, and differentiation studies because nearly all tissue cells in vivo are embedded in a 3-D extracellular microenvironment with a complex and dynamic molecular composition. Artificial 2-D substrates are likely to misrepresent findings by forcing cells to adjust to flat and rigid surfaces unlike the in vivo environment. For that reason, varying degrees of 3-D cell culture substrates, with properties between 2-D Petri dishes and in vivo mouse models, have been developed with various bio- and synthetic-polymers.

Numerous studies have shown that a 3-D cell culture system offers a more realistic micro- and local-environment where the functional properties of cells can be observed and manipulated. However, there is no standard 3-D cell scaffold because of the variability of scaffolds resulting from existing scaffold-manufacturing techniques. Current scaffold-fabricating technologies, which can include porogen leaching, freeze-drying, and gas foaming, produce highly porous structures with stochastically arranged pores. The resultant scaffolds lack precision in the shape and dimension of pores and channels, surface chemistry, and mechanical properties, leaving the experimentalist without control over the 3-D cellular microenvironment. To obtain results that mimic the in-vivo cellular response and are highly reproducible, one requires a 3-D scaffold with precisely controlled properties. The present disclosure is a standard method for fabricating 3-D inverted colloidal crystal (ICC) scaffolds that fit directly into standard cell culture well plates, including 96-well microplates, with highly controllable macro-, micro- and nano-scale properties, minimizing product variability and experimental results. By making the ICC cell scaffold size fit to a cell culture well microplate, this new type of 3-D cell scaffolds can be easily accepted in the current research field.

Driven by the desire of the pharmaceutical industry to replace some portion of regular in vitro drug testing and potentially some animal trials with experiments in 3-D cell cultures, which can reduce the cost and the time from inception to production of a new drug, much work is also being done on the development of replicas of in vivo cellular structures.

Numerous materials and manufacturing processes have been tested to create 3-D scaffolds for 3-D in vitro drug testing studies. However, many of them are impractical for mass drug testing due to strong light scattering/absorption and variability in the scaffold quality/topology. Virtually all of the commercially made 3-D scaffolds are made from ceramics or other inorganic materials and are difficult to incorporate in established drug evaluation protocols. Despite recent advances in tissue engineering and automation of biological systems, it would be useful to provide structures and automated methods of making biomimetic structures capable of growth and maintenance of cultured cells under controlled conditions to study the effects of biologically active molecules including hormones, growth and differentiation factors, cytokines, pharmaceuticals, enzymes, toxins, antigens and biological organisms.

Further areas of applicability will become apparent from the description provided herein. It should be understood that the description and specific examples are intended for purposes of illustration only and are not intended to limit the scope of the present disclosure.

### SUMMARY

The present disclosure provides an improved three dimensional inverted colloidal crystal scaffold comprising a cell culture plate having at least one well and a three dimensional matrix comprising a transparent biocompatible polymeric hydrogel network containing microspherical voids. The microspherical voids are each connected to at least one other void through inter-connecting pores.

In another aspect, the present disclosure provides a method of making an inverted colloidal crystal scaffold, the method comprising: providing a substrate comprising one or more wells and introducing into each of the wells a plurality (more than one) of microspheres into each well forming a colloidal crystal template of the plurality of microspheres. The colloidal crystal template comprises a plurality of microspheres and between the microspheres interstitial spaces therebetween. The colloidal crystal template consisting of layered microspheres are heated to partially melt the microspheres and form junctions with each other. The colloidal crystal template is then contacted with a biocompatible polymer precursor around the microspheres filling the interstitial spaces. The polymer precursor is then polymerized to form an integrated transparent three dimensional polymer network. The microspheres within the polymer network are removed thereby forming an inverted colloidal crystal scaffold comprising a transparent biocompatible polymer network with interconnected spherical voids.

An automatic ICC scaffold apparatus is also described, as are methods for using the ICC scaffold for culturing cells and for identifying the effects of a compound on cell function using the ICC scaffolds containing living cells.

There are several advantages of the present teachings. First, the 3-D ICC scaffolds of the present disclosure afford advantages relating to greater mass transport of nutrients and gasses over the continuous 3-D scaffolds previously shown. Second, the present ICC scaffolds can be made transparent which greatly facilitates monitoring and analysis of cells when incubated in experimental test conditions over other opaque 3-D matrix scaffolds. Third, cells seeded within the 3-D ICC scaffold exhibit greater wild-type activity over 2-D artificial constructs, and does not impede outgrowth of cell processes in three dimensions. Fourth, the interconnected pores within the spherical cavities permit communication between cells and diffusion of nutrients and gasses to even the interior of the scaffold permitting true cell colonization and wild type cell function. Fifth, the present disclosure describes high throughput cell studies and assays performed in a 3-D microenvironment in the same way that 2-D studies are performed in cell culture well microplates. The automated system of producing 3-D hydrogel cell scaffolds in cell culture well microplates is believed novel, as is an automated system of producing ICC scaffolds. Sixth, the described ICC scaffolds can be conveniently made in the well-plate format, while the other types of 3D scaffolds, such as bone-like scaffolds from inorganic matrix cannot be easily fit into the wells due to brittleness of the material.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIGS. 1A-1C are scanning electron micrographs of various sized templated micropsheres layered in a highly ordered array. FIG. 1C-1D are scanning electron micrographs of inverted colloidal crystal scaffolds after the micropsheres have been removed revealing the three dimensional polymer network wherein the voids or cavities are interconnected with pores

FIG. 2 is an illustration of one pattern of deposition of uniformly sized microspheres in a substrate comprising one well depicting the formation of a hexagonal array.

FIG. 3 is a side elevational view of an automated apparatus for the fabrication of inverted colloidal crystal scaffolds in accordance with the present disclosure

FIGS. 4A and 4B are photographs of the ICC colloidal crystal scaffold layered in a 96-well microplate substrate. The photograph is showing the top of the microplate. FIG.4B shows the bottom image of the 96-well microplate

FIG. 5 is a photograph of 96-well microplate containing ICC scaffolds of the present disclosure after covering with a transparent sealing tape.

FIG. 6 is an illustration of a substrate arrangement containing an ICC scaffold layered on a membrane with culture media recirculating channels to circulate media throughout the scaffold.

### DETAILED DESCRIPTION

The following description is merely exemplary in nature and is not intended to limit the present disclosure, application, or uses. In accordance with the present disclosure, it has been found that inverted colloidal crystal (ICC) scaffolds comprising a biocompatible three-dimensional matrix of hydrogel can be manufactured to sustain and promote the growth and differentiation of living cells conveniently produced in tissue culture plates, including microplates. The present disclosure described herein further describes methods for the automated system of fabricating ICC scaffolds for standard cell culture plates, including without limitation, microplate tissue culture plates for use in assays relating to: cell-biology, toxicology, pharmacology, biochemistry, molecular biology, immunology and pathology.

In some embodiments, cells can be seeded, grown and manipulated in the ICC scaffolds using established cell-biology protocols commonly known in the art. The ICC scaffolds can be designed to advance current biological fields, including cell biology, biochemistry, molecular biology, microbiology, and systems biology. For example, cell culture well microplates are commonly used in stem cell biology studies to perform multiple experiments using a limited number of stem cells. Additionally, research has shown that a 3-D culture environment can significantly reduce or eliminate the use of expensive cytokines that are necessary in 2-D stem cell cultures. Because the differentiation of stem cells can be highly influenced by signals from the 3-D environment, a uniform and highly controlled 3-D substrate within each well on the cell culture well microplate will improve economically current stem cell research techniques.

### ICC Scaffolds

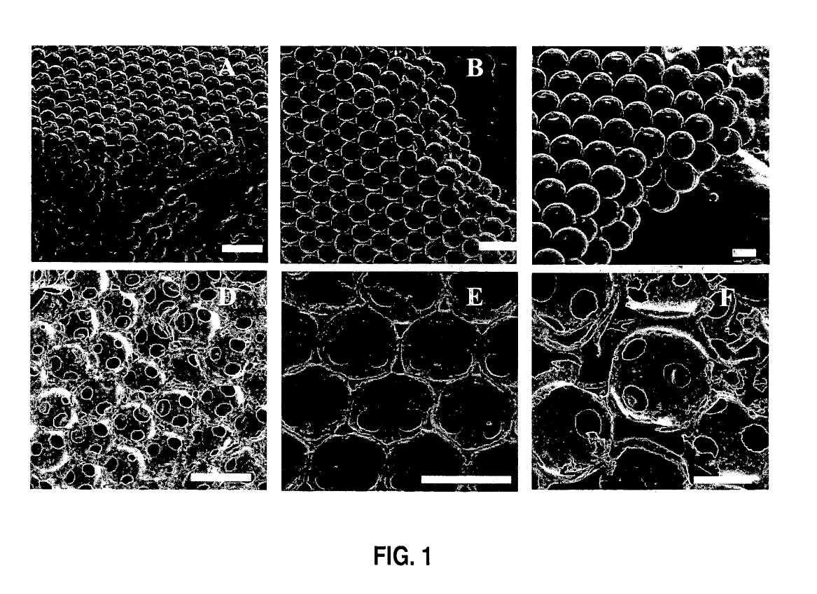

In some embodiments, the three dimensional inverted colloidal crystal scaffold comprises a substrate having at least one well and a three dimensional polymer matrix comprising a transparent polymer network having a plurality of empty spherical cavities having interconnected pores arranged in a hexagonal crystal lattice See FIG. 1A-1C. As shown in FIG. 1, the ICC scaffolds comprise a transparent 3-D polymer matrix containing a porosity consisting of voids or cavities having one or more interconnected pores between adjacent voids. In some embodiments, the voids are seeded with cells to form a transparent polymer ICC cell scaffold. As used herein, the 3-D polymer matrix can comprise any transparent, biocompatible polymer including for example, polystyrene, collagen gel, fibrin gel, poly(lactic acid), polypeptides, as well as co-polymers of these compounds, hydrogels, bioglasses or inorganic gels. The ICC scaffold can be placed in any substrate including without limitation, any suitable tissue culture plate having at least one well with at least one generally planar surface. In some embodiments, the substrate is a microplate having 48, 96, 384 or 1536 wells. In some embodiments, ICC scaffolds can be manufactured in cell culture plates having a plurality of wells ranging from 2 to 1536 identical or different sized wells. In some embodiments described herein, the ICC scaffolds can be manufactured and utilized to fit the wells of a cell culture well microplate (e.g. 24, 48, 96 384, or 1536 wells) to improve and standardize the cell growth environment of existing experiments, without significantly altering the procedures and materials required by the scientist.

In some embodiments, the ICC scaffold comprises a cell culture plate having at least one well comprising a planar surface disposed within the well. Generally, the substrate can be any commonly used cell-culture material that is inert and biocompatible, for example plastics, glass, ceramic, metallic and combinations thereof. In non-limiting examples, the substrate containing wells within for example, of the microplates, can comprise polypropylene, polyethylene terephthalate, polytetrafluoroethylene, polyaryletherketone, nylon, fluorinated ethylene propylene, polybutylester, silicone or combinations thereof. In some embodiments, cell culture plates and wells therein, can include for example, one well cell culture plate (square or round Petri dish), 2 well cell culture dish, 4 well cell culture dish, 8 well cell culture dish, 12 well cell culture dish, 24 well cell culture dish, 48 well cell culture dish, 96 well cell culture microplate, 384 well cell culture microplate and 1536 well microplate. The cell culture plates, dishes, or microplates can be made of polypropylene, polycarbonate, polystyrene and other commonly known tissue culture plastic. In some embodiments, the cell culture plate has a flat-bottomed well, meaning that the surface upon which the ICC scaffold is made contains a substantially planar surface having a wall generally made of the same material orthogonal to the plane of the surface capable of containing a predetermined volume of liquid containing a hexagonal array of microspheres.

In some embodiments, the ICC scaffolds can comprise polymers that are biocompatible including polymers that impart both high transparency and elasticity. In some embodiments, the polymer can be a hydrogel. Hydrogels may be formed from covalently or non-covalently crosslinked materials, and may be non-degradable (“biostable”) in a physiological environment or broken down by natural processes, referred to as biodegradable or bioabsorbable. The hydrogels generally exclude silica or metallic polymer matrices. The breakdown process may be due to one of many factors in the physiological environment, such as enzymatic activity, heat, hydrolysis, or others, including a combination of these factors.

Hydrogels that are crosslinked can be crosslinked by any of a variety of linkages, which may be reversible or irreversible. Reversible linkages can be due to ionic interaction, hydrogen or dipole type interactions or the presence of covalent bonds. Covalent linkages for absorbable or degradable hydrogels can be chosen from any of a variety of linkages that are known to be unstable in an animal physiological environment due to the presence of bonds that break either by hydrolysis (e.g., as found in synthetic absorbable sutures), enzymatically degraded (e.g., as found in collagen or glycosamino glycans or carbohydrates), or those that are thermally labile (e.g., azo or peroxy linkages).

All of the hydrogel materials appropriate for use in the present disclosure should be physiologically acceptable and should be swollen in the presence of liquid, including water and tissue culture media. The hydrogel can be formed by polymerization of monomer precursor solution in the well of the substrate.

In some embodiments, hydrogels can be formed from natural, synthetic, or biosynthetic polymers. Natural polymers can include glycosminoglycans, polysaccharides, proteins etc. The term “glycosaminoglycan” is intended to encompass complex polysaccharides which are not biologically active (i.e., not compounds such as ligands or proteins) and have repeating units of either the same saccharide subunit or two different saccharide subunits. Some examples of glycosaminoglycans include dermatan sulfate, hyaluronic acid, the chondroitin sulfates, chitin, alginate heparin, keratan sulfate, keratosulfate, and derivatives thereof.

In general, the glycosaminoglycans can be extracted from a natural source and purified and derivatized. However, they also may be synthetically produced or synthesized by modified microorganisms such as bacteria. These materials may be modified synthetically from a naturally soluble state to a partially soluble or water swellable or hydrogel state. This modification can be accomplished by various well-known techniques, such as by conjugation or replacement of ionizable or hydrogen bondable functional groups such as carboxyl and/or hydroxyl or amine groups with other more hydrophobic groups.

The polymerizable hydrogels are made by polymerizing either through photo-curing, actinic radiation (UV, ion-beam and other ionizing radiation), or by cross-linking hydrogel monomers (including chemical, enzymatic and glycation). Hydrogels can be polymers, homopolymers, heteropolymers, co-polymers and block co-polymers. Suitable hydrogels can include, but are not limited to, aminodextran, dextran, DEAE-dextran, chondroitin sulfate, dermatan, heparan, heparin, chitosan, polyethyleneimine, polylysine, dermatan sulfate, heparan sulfate, alginic acid, pectin, carboxymethylcellulose, hyaluronic acid, agarose, carrageenan, starch, polyvinyl alcohol, cellulose, polyacrylic acid, poly(meth) acrylates, poly meth(methacrylate) PMMA, polyacrylamide, polyhydroxyalkanoates (PHA and PHB), polycaprolactone, polyetheretherketone polyglycolidepoly-3-hydroxybutyrate, polyethylene glycol, or the salt or ester thereof, or a mixture thereof.

Synthetic polymeric hydrogels generally swell or expand to a very high degree, usually exhibiting a 2 to 100-fold volume increase upon hydration from a substantially dry or dehydrated state. Synthetic hydrogels may be biostable or biodegradable or bioabsorbable. Biostable hydrophilic polymeric materials that form hydrogels useful for practicing the present disclosure include poly(hydroxyalkyl methacrylate) including poly(meth) methacrylates, poly(electrolyte complexes), poly(vinylacetate) cross-linked with hydrolysable bonds, and water-swellable N-vinyl lactams. The swellable hydrogel can be used in manufacturing of the well-containing scaffolds by placing unswelled state of the hydrogel into the scaffold and transferring it to the swollen state to fit tightly in the well. In some embodiments, swellable hydrogels can be used for cell extraction from the scaffolds. The scaffold with attached cells can be placed in a media inducing swelling and the expansion of the hydrogel causing the detachment and release of the cells into the media.

Other suitable hydrogels can include hydrophilic hydrogels know as CARBOPOL.®., a registered trademark of B. F. Goodrich Co., Akron, Ohio, for acidic carboxy polymer (Carbomer resins are high molecular weight, allylpentaerythritol-crosslinked, acrylic acid-based polymers, modified with C10-C30 alkyl acrylates), polyacrylamides, such as those marketed under the CYANAMER.®. name, a registered trademark of Cytec Technology Corp., Wilmington, Del., polyacrylic acid marketed under the GOOD-RITE.®. name, a registered trademark of B. F. Goodrich Co., Akron, Ohio, polyethylene oxide, starch graft copolymers, acrylate polymer marketed under the AQUAKEEP.®. name, a registered trademark of Sumitomo Seika Chemicals Co., Japan, ester crosslinked polyglucan, and the like. Such hydrogels are described, for example, in U.S. Pat. No. 3,640,741 to Etes, U.S. Pat. No. 3,865,108 to Hartop, U.S. Pat. No. 3,992,562 to Denzinger et al., U.S. Pat. No. 4,002,173 to Manning et al., U.S. Pat. No. 4,014,335 to Arnold and U.S. Pat. No. 4,207,893 to Michaels, all of which are incorporated herein by reference, and in Handbook of Common Polymers, (Scott & Roff, Eds.) Chemical Rubber Company, Cleveland, Ohio.

Hydrogels can also be formed to be responsive to changes in environmental factors, such as pH, temperature, ionic strength, charge, etc., by exhibiting a corresponding change in physical size or shape, so-called “smart” gels. For example, thermoreversible hydrogels, such as those formed of amorphous N-substituted acrylamides in water, undergo reversible gelation when heated or cooled about certain temperatures (lower critical solution temperature, LCST). Prevailing gel formation mechanisms include molecular clustering of amorphous polymers and selective crystallization of mixed phases of crystalline materials. Such gels, which are insoluble under physiological conditions, also advantageously can be used for practicing the present disclosure.

It is also possible to affect the rate at which a substantially dehydrated hydrogel rehydrates in a physiological environment, such as encountered upon implantation in an animal. For example, creating a porous structure within the hydrogel by incorporating a blowing agent during the formation of the hydrogel may lead to more rapid re-hydration due to the enhanced surface area available for the water front to diffuse into the hydrogel structure.

The hydrogel forming precursors for the foregoing ICC scaffolds can be selected so that, for example, a free radical polymerization is initiated when two components of a redox initiating system are brought together.

In addition, the driving force for water to penetrate a dehydrated hydrogel also may be influenced by making the hydrogel hyperosmotic relative to the surrounding physiological fluids. Incorporation of charged species within hydrogels, for example, is known to greatly enhance the swellability of hydrogels. Thus the presence of carboxyl or sulfonic acid groups along polymeric chains within the hydrogel structure may be used to enhance both degree and rate of hydration. The surface to volume ratio of the implanted hydrogels also is expected to have an impact on the rate of swelling. For example, crushed dried hydrogel beads are expected to swell faster to the equilibrium water content state than a rod shaped implant of comparable volume.

Any of a variety of techniques may be used to form hydrogels in the cell culture plate or microplate. For example, monomers or macromers of hydrogel forming compositions can be further polymerized to form three dimensionally cross-linked hydrogels. The crosslinking may be covalent, ionic, and or physical in nature. Polymerization mechanisms permitting in-situ formation of hydrogels are per se known, and include, without limitation, free radical, condensation, anionic, or cationic polymerizations. The hydrogels also may be formed by reactions between nucleophilic and electrophilic functional groups, present on one or more polymeric species, that are added either simultaneously or sequentially. The formation of hydrogels also may be facilitated using external energy sources, such as in photoactivation, by UV light, thermal activation and chemical activation techniques.

Polymer precursors used to make the ICC scaffold, including hydrogels, can be fluorecscently labeled during the polymer synthesis or after polymerization to facilitate imaging processing of the cells contained in the ICC scaffolds. The fluorescent labeling can involve addition of specific dyes to the hydrogel composition or specific fluorescent groups to the monomer(s) in the polymerization process. The dyes can be covalently, iononically, cooperatively, hydrophobically or otherwise bonded, for instance using hydrogen, donor-acceptor, van-der Waals, bonds, to the hydrogel matrix.

Synthesis and biomedical and pharmaceutical applications of absorbable or biodegradable hydrogels based on covalently crosslinked networks comprising polypeptide or polyester components as the enzymatically or hydrolytically labile components, respectively, have been described by a number of researchers. See, e.g., Jarrett et al., “Bioabsorbable Hydrogel Tissue Barrier: In Situ Gelation Kinetics,” Trans. Soc. Biomater., Vol. XVIII, 182 (1995); Sawhney et al., “Bioerodible Hydrogels Based on Photopolymerized Poly(ethyleneglycol)-co-poly(.alpha.-hydroxy acid) Diacrylate Macromers”, Macromolecules, 26:581-587 (1993); Park, et al., Biodegradable Hydrogels for Drug Delivery, Technomic Pub. Co., Lancaster, Pa. (1993); Park, “Enzyme-digestible swelling hydrogels as platforms for long-term oral delivery: synthesis and characterization,” Biomaterials, 9:435-441 (1988). The hydrogels most often cited in the literature are those made of water-soluble polymers, such as polyvinyl pyrrolidone, which have been crosslinked with naturally derived biodegradable components such as those based on albumin.

Totally synthetic hydrogels have been studied for controlled drug release and membranes for the treatment of post-surgical adhesion. Those hydrogels are based on covalent networks formed by the addition polymerization of acrylic-terminated, water-soluble polymers that have at least one biodegradable spacer group separating the water soluble segments from the crosslinkable segments, so that the polymerized hydrogels degrade in vivo. Such hydrogels are described in U.S. Pat. No. 5,410,016, which is incorporated herein by reference, and may be particularly useful for practicing the present disclosure.

Thus, hydrogels suitable for use in the present disclosure preferably are physically or chemically crosslinked, so that they possess some level of mechanical integrity even when fully hydrated. The mechanical integrity of the hydrogels may be characterized by the tensile modulus at breaking for the particular hydrogel. Hydrogels having a tensile strength in excess of 10 KPa are preferred, and hydrogels having a tensile strength greater than 20 KPa are more preferred. In some embodiments, biocompatible hydrogels can be used in polymerizable and non-polymerizable forms.

The hydrogel can be used as-is or further modified depending upon the desired use of the ICC scaffold. For example, the hydrogel can be derivatized with one or more different chemical groups so that the hydrogel can form bonds with other chemicals applied to the hydrogel, for example a polyelectrolyte chemical layer. In some embodiments, a polyelectrolyte can form a non-covalent or covalent bond with the hydrogel.

In some embodiments, the hydrogel can be transparent after polymerization. In some embodiments, high transparency of the ICC scaffold can be maintained even after the hydrogel is coupled with various chemical layers, biological molecules and cells. Transparency of the hydrogel permits the optical assessment of cell growth, presence of colored, fluorescent, luminescent, opalescent, phosphorescent markers and binding agents. In some embodiments, the final transparency of the ICC scaffolds can be measured using any commonly known objective measurement of transparency in plastics, containers and bottles. The method to measure transparency can be directed to measuring human perception of transparency by measuring total transmittance, transmission, haze and clarity for example using American Society for Testing and Materials (ASTM), standard ASTM D1746-03.

### Methods of Making the ICC Scaffold and Hydrogel ICC Cell Scaffolds

In some embodiments, the ICC scaffolds are manufactured by first making a hexagonal array of microspheres or beads in solution as shown in FIG. 1(A-C). Once the microspheres have settled on the well surface in their lowest energy conformation (FIG. 1A-C), the microspheres can be heated sufficiently to melt, creating at the contact junctions with other microspheres. The microspheres are then cooled and templated with a solution of hydrogel. The microspheres are templated by adding a solution comprising one or more types of hydrogel monomer into and around the array to fill in the interstitial spaces and thus templating the microsphere hexagonal array to produce a 3-D hydrogel matrix. Upon polymerization and/or hardening, the microspheres are dissolved in a solvent thus leaving an inverted colloidal hydrogel scaffold containing cavities with interconnecting pores where the microspheres are connected to one another as shown in FIG. 1(D-F).

In some embodiments, the preparation of ICC scaffolds from hydrogel is carried out in five steps: (1) self-assembly of colloidal crystals from monodispersed micron-scale glass, PMMA, polystyrene or latex spheres by sedimentation; (2) annealing of the primary colloidal crystal mold to obtain rigidity of the structure and desirable diameter of the interconnecting channels; (3) application of hydrogel into the interstitial spaces between the arrayed microspheres/infiltration and curing; (4) removal of the glass, PMMA polystyrene or latex microspheres or beads by dissolving them in solvent; and (5) thorough washing the 3-D porous hydrogel matrix with PBS buffer. Several benefits are imparted by such a preparation procedure, including the use of a hydrogel matrix, which does not require a high temperature for its production. Other advantages can include reducing the cost of manufacturing, increasing the quality, reproducibility, stability and biocompatibility of these scaffolds. These steps will be further exemplified below.

### Colloidal Crystal Construction

To utilize the unique geometry of ICCs as a cell scaffold the cavity size, and thus microsphere size, can be within the 50-1000 μm range. Possible strategies for constructing highly packed micro-scale colloidal crystals can include retardation of microsphere sedimentation rate and gentle agitation. These strategies can be achieved utilizing two distinct properties that micro-sized spheres possess over nano-sized spheres: effective agitation of larger volume spheres by shear force, and faster sedimentation rate of heavier spheres. In some embodiments, the microspheres can be made from any material that can form spherical bodies and which can partially melt or anneal to form junctions at the point of contact with other microspheres. In some embodiments, the microspheres comprise glass, for example, soda-lime glass, (or other glasses comprising mixtures of silicon dioxide, sodium carbonate, and either calcium carbonate or calcium oxide which can be dissolved without dissolution of the hydrogel matrix), latex particles, poly(styrene) and the like.

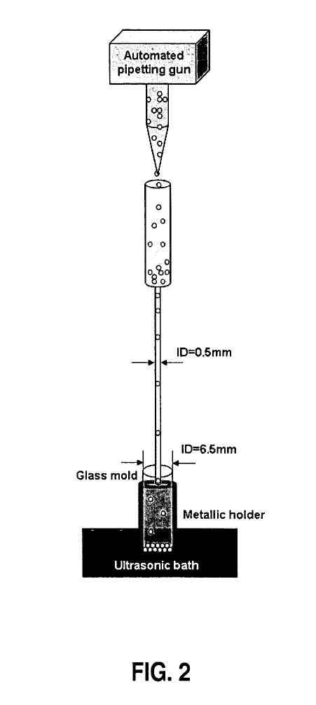

Microspheres can be introduced into a Pasteur pipette before entering into the cell-culture plate well/mold to extend the sedimentation distance. In doing so, the pipette works as a thin funnel causing a bottleneck effect for precipitating microspheres as show in FIG. 2. In some embodiments, injected microspheres can sediment one at a time. As microspheres precipitate to the bottom of the mold, gentle agitation generated by an ultrasonic bath can assist the movement of microspheres enabling the microspheres to be positioned on the substrate in their lowest energy configuration. In some embodiments, the microspheres of equal or substantially equal size can be highly packed and ordered as shown in FIGS. 1A and 1B. In some embodiments using microspheres of equal or substantially equal size, a hexagonal array can be formed according to the methods of the present disclosure as shown in FIG. 1. In some embodiments, other geometrical arrangements can be formed by allowing microspheres of different sizes to be closely packed together forming contact points with adjacent microspheres. In some embodiments, each microsphere can contact six or more microspheres positioned in three dimensions. Each layer of microspheres can serve as a template for the formation of the next layer, so when microspheres are added drop-by-drop the entire resulting structure can be seen to include microspheres having the same or substantially the same number of contacts with other microspheres as illustrated in FIG. 1(D-F).

In some embodiments, following preparation of highly packed colloidal crystals, the colloidal crystals can be heat-treated to partially melt the spheres. Upon slight melting, junctions are formed at points of contact between microspheres. As spheres are cooled, the junctions set, creating a solid colloidal structure. The resulting free-standing colloidal crystals are strong enough to be easily handled and removed from the well/mold. The formation of junctions prevents breakage of the crystal lattice during the infiltration of scaffolding material and ensures continuity of the chain of pores in the final scaffold. The channel or pore diameter is determined at this stage because the size of melted area depends on the annealing temperature.

In some embodiments, uniformly sized soda lime glass microspheres, having diameters ranging from 50-500 μm, can be used to make colloidal crystals. As a substrate, flat bottom cylindrical borosilicate glass shells can be employed, because of the higher softening temperature of borosilicate. To retard the precipitation rate, ethylene glycol can be used as a medium or solvent. In some embodiments, the diameters of poly(meth) methcrylate (PMMA) and glass beads commercially available for the preparation of ICC scaffolds can vary widely, depending on the desired application of the ICC scaffold. In some embodiments, the microspheres can range from about 50 μm to about 100 μm, from about 50 μm to about 200 μm, from about 50 μm to about 300 μm, from about 50 μm to about 400 μm, from about 50 μm to about 500 μm, from about 500 μm to about 400 μm, from about 500 μm to about 300 μm, from about 500 μm to about 200 μm, from about 500 μm to about 100 μm, and from about 500 μm to about 100 μm. In some embodiments, the colloidal crystals can be assembled by slow sedimentation of microspheres with diameters of 50, 100, 150, 200, 250, 300, 400, and 500 microns in water.

Aqueous solvent mixtures with glycerol or ethylene glycol can be used to slow down the sedimentation of microspheres and increase the geometric perfection of the scaffolds. Increasing the amount of glycerol can decrease of the speed of sedimentation and can improve the degree of order of the colloidal crystals. The same effect can also be achieved by manipulating pH and ionic strength in aqueous solutions. Increasing the electrostatic repulsion between the negatively charged beads can slow down their precipitation process and decrease van-der Waals attraction that typically results in defects. Reduction of ionic strength and elevating pH from about 7.5 to, about pH 9.0 can result in stronger electrostatic forces between the beads, thus promoting a more highly ordered array.

Infiltration of colloidal crystal molds with hydrogel. For each bead size between 50 and 500 microns, annealing of the primary colloidal crystal can be performed to impart sturdiness to the colloidal crystal mold and to create bridges between the spheres, which eventually become interconnecting poles. For poly(meth) methacrylate (PMMA), the temperature of annealing, Tₐₙₙ, can vary between 80° C. and 150° C. with an interval of 100° C., and can also vary the time of annealing, tₐₙₙ. For glass beads, Tₐₙₙ can vary between 660° C. and 850° C. with an interval of 190° C. The higher the temperature of annealing, Tₐₙₙ, and the longer the corresponding time of the process, tₐₙₙ, the more pronounced the bridging (pore interconnection) will be. Based on these two parameters, Tₐₙₙ and tₐₙₙ, a calibration table can be constructed that can allow a skilled practitioner to control the geometry of the scaffolds. In some embodiments, calculating the appropriate annealing temperatures and time of annealing can allow one skilled in the art to manufacture a wide array of scaffolds for individual applications and result in customizable ICC scaffolds for varying cell growth conditions.

Other methods of consolidation of microspheres under external stimulus or stimuli can be applied as well, and can include photochemical, microwave, magnetic, physical treatment and other stimuli. In some embodiments, no external stimuli may be applied.

In some embodiments, annealing can be followed by infiltration with one or more hydrogel compositions, for example, poly(acrylamide) or alginate hydrogel. In some embodiments, the hydrogel preparation can comprise one or more polymerization methods to synthesize the hydrogels. In the example of polyacrylamide hydrogels can proceed by the addition of thermo-initiation, 10 μL of 2% K₂S₂O₈ and 0.1 mL of water being added to 0.5 mL of degassed hydrogel monomer solution, i.e. 30% w/w acrylamide monomer with various amount of a cross-linking agent, for example multifunctional crosslinkers such as ethylene glycol dimethacrylate (EGDMA), N,N′ methylenebisacrylamide (NMBA), 1,4 butanediol dimethacrylate (BDMA) and trimethylolpropane triacrylate (TMPTA) as cross-linking agent. The mixture can be infiltrated into the primary colloidal array and then polymerized for varying temperatures and times, depending on the percentage monomer and concentration of cross-linking agent. In some embodiments, the acrylamide hydrogel can be polymerized at 70° C. for 12 hours. For redox initiation, 0.5 mL of monomer solution, 0.1 mL of 0.05M L-ascorbic acid and 10 μL of 2% K₂S₂O₈ can be mixed. The hydrogel mixture can be infiltrated into the primary colloidal crystal array, and polymerization can be carried out to completion at room temperature for 12 hours. The resulting gel can then be soaked in tetrahydrofuran (THF) to remove the polymeric colloid array comprising the microspheres. The inverted hydrogel scaffold can then soak in water and can reach an equilibrium swelling state at room temperature.

In some embodiments, the glass beads can be removed by soaking in 0.5% hydrofluoric acid (HF) with subsequent thorough rinsing to dissolve the glass beads and leave the polymerized hydrogel matrix intact. Wash steps can be employed to remove the HF until the concentration of F-falls below the concentration of fluoride in de-ionized water (approximately <10⁻⁵M). After the cavities have been formed by dissolving the glass microsphere in the hydrogel, the cavities can be expected to have between 3 to about 12 pores per spherical cavity. The hydrogel matrix can comprise from about 50% to about 90% porosity by volume of the matrix.

The geometrical characteristics of the hydrogel scaffolds can be evaluated and verified using confocal microscopy in addition to environmental scanning electron microscopy (SEM), which does not cause drying of the hydrogel. The diameters of spherical cavities formed in place of the microspheres and the diameters of interconnecting pores formed in place of interparticle contact junctions can be measured and compared to the parameters of the original colloidal particles. The empirical dependence between Tₐₙₙ and tₐₙₙ and the diameter of interconnecting pores can be determined and selected, ranging in size between 50 and 500 nm.

### Automatic Colloidal Crystal Construction System

In another embodiment, the present disclosure is directed to an apparatus for the use in the production of ICC scaffolds and hydrogel ICC cell scaffolds comprising a 3-D porous ICC scaffold having cavities, wherein the cavities each have interconnecting pores as described herein.

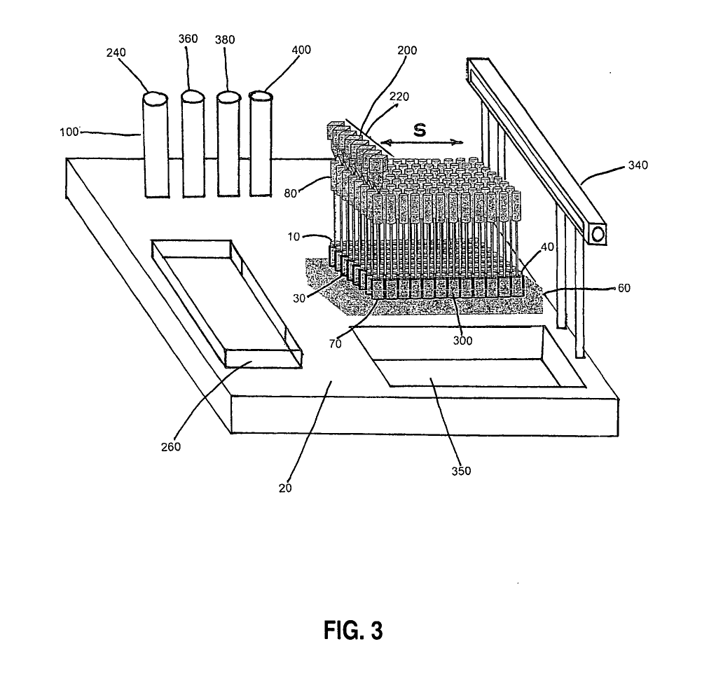

The apparatus of the present disclosure can be described with reference to FIG. 3. In FIG. 3, an apparatus for producing ICC scaffolds having a porous hydrogel 3-D matrix is illustrated. The apparatus comprises a commercially available glass vial well plate 10 operably mounted on the surface of a table 20. The glass vial well plate 10 consists of a metal base 30 with spaces to fix cell culture flat bottom glass vials 40 in 12 rows of 8 vials. The glass vials 40, with inner diameters ranging from 5-7 mm, possess the same dimensions as wells in a standard cell culture well microplate, and serve as molds for colloidal crystals. The glass vial well plate 10 sits in an ultrasonic bath 60 mounted on the table 20, so that the bottom ends of the holders 70 are submerged in the bath.

A plurality of dispensers, for example, without limitation Pasteur pipettes 80 can be secured to each glass vial 40 to ensure slow sedimentation of microspheres into the mold. The Pasteur pipette 80 can be centered in the opening of each vial 40, and placed so that its tip is within the vial. The pipette 80 and vial 40 can be filled with ethylene glycol obtained from one of a plurality of reservoirs 100 to allow for slow sedimentation of microspheres through the pipette 80 and into the vial 40.

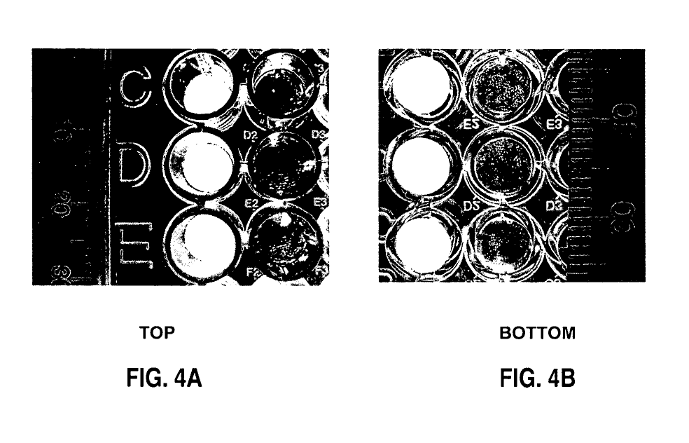

A uniform quantity of glass microspheres is preferably injected into each mold. To obtain uniform microsphere distribution, an automated microplate pipetting system 200 is used to deliver accurate volumes of microspheres, reagents, hydrogel solution and wash solutions. An automated microplate pipetting system 200 consists of 8 micropipette tips aligned in a row 220, spaced identically as the 8 wells in each row of a cell culture well plate, for example in microplate 10. The automated microplate pipetting system 200 is positioned above the Pasteur pipettes 80 and vials 40 so that a consistent quantity of glass microsphere dispersion from a microsphere reservoir 240 is dropped simultaneously in each of the eight Pasteur pipettes 80 in a row. After simultaneously releasing a drop of microspheres into each of the 8 pipettes 80, the automated system moves to the next row. This is repeated for each of the 12 rows, and then the automated system is timed to rest for 15 minutes before dispersing another drop in each pipette. The apparatus can also comprise a timing means such as an electronic, digital or analog timing mechanism to actuate the various components, including the automated microplate pipetting system 200, the ultrasonic bath 60, and the oven 260 and alarm systems not shown. A 15-minute gap between each drop release can be designed to ensure microspheres sediment slowly and find their lowest energy configuration, forming a hexagonal close-packed array 300, before the next drop is added. Once the colloidal crystal array has reached the desired height (from about 0.3 to about 1.5 mm), Pasteur pipettes 80 are removed, and microplates 20 are left under gentle agitation in the ultrasonic water bath 60 for 4-5 hours without further addition of microspheres as shown in FIG. 4A and FIG. 4B.

### Automatic Drying and Annealing System

The glass vial well plate containing cell culture molds is transferred either manually or robotically to an oven 260 preset to a temperature ranging from about 120° C. to about 170° C. for about 10-15 hours to evaporate all solvent, leaving dry, un-annealed colloidal crystals. The temperature can be gradually increased to a range from about 660° C. to about 850° C., depending on the size of microspheres, for about 2-3 hours to anneal the microspheres together, forming a solid colloidal crystal array. The solid colloidal crystal array can serve as a template for the ICC. The oven temperature can be set and changed by a timer.

### Automatic Hydrogel Infiltration and Polymerization System

The glass vial well plate 10 can be removed from the oven manually or robotically and placed on the apparatus table 20 or ultrasonic bath 60 for further liquid manipulation steps described herein. The automated microplate pipetting system 200 injects a hydrogel precursor solution into the vials 40 containing colloidal crystals, under slight agitation in the ultrasonic bath 60 to ensure complete infiltration. Once the hydrogel precursor solution has filled the entire volume of the colloidal crystal, the colloidal crystals can be removed from the molds and put between two highly absorbable sponge sheets. By briefly pressing down on the colloidal crystals from opposite directions, precursor solution at the top and bottom of colloidal crystals can be effectively removed; precursor solution remains in the inner space or interstitial spaces of colloidal crystals by capillary force. Next, colloidal crystals are exposed to UV light 340 for 12 hours to polymerize the hydrogel precursor solution.

### Automatic Microsphere Dissolving and Washing System

The colloidal crystals infiltrated with polymerized hydrogel can be transferred to a plastic bath 350 on the apparatus table 20 containing a solution derived from reservoir 360 containing for example, 1% HF, using an automated liquid dispensing means operably connected to a power source and pump to retrieve solution from one or more of the plurality of reservoirs 100. The colloidal crystals can be stirred periodically or continuously for approximately 2 days using an automatic stirrer such as a magnetic stirrer. The automated pipetting system 200, can periodically remove solution from the plastic bath 350 and replace the retrieved solution with fresh HF solution obtained from reservoir 360 in an equal or different volume. The washing system is designed to continuously remove and replenish 1% HF. After microspheres are dissolved from the hydrogel, an inverted replica of the colloidal crystal remains, which is a hydrogel ICC. Hydrogel ICCs can be removed from the 1% HF solution, and placed into a circulating bath 350 of deionized water for 24 hours, which is obtained using the automated pipetting system 200, from reservoir 380. Water is removed and then the hydrogel ICCs can be washed in a solution of phosphate buffered saline contained in reservoir 400 to neutralize any remaining HF using the automated pipetting system 200. The ICCs can then be rinsed again in deionized water obtained from reservoir. In some embodiments the ICC scaffolds can be made by cutting out large sheets or cylinders of ICC scaffold matrix made by self-organization of colloidal spheres followed by their infiltration with biocompatible polymer precursor for example a hydrogel polymer precursor, followed by removal of the beads. Cutting from a large piece of the hydrogel matrix will significantly accelerate the production process and will allow one to reduce the time and cost to prepare the scaffolds.

### ICC Scaffold Surface Coating Through the Layer-by-Layer (LBL) Method

In some embodiments, methods are described to functionalize the surface of the ICC scaffold using a layer-by-layer (LBL) approach. The LBL method can be adaptable to any chemical process and allows functionalization of the scaffolds with any kind of biocompatible material individually, sequentially or as a mixture following virtually the identical procedure while retaining their biological activity.

In some embodiments, the LBL method is also known as polyelectrolyte multilayers (PEM) and electrostatic self-assembly. In some embodiments, the LBL method comprises sequential dipping of a substrate having contained therein an ICC scaffold into a solution of oppositely charged species alternating with water rinse. The first rinse can be any charged polyelectrolyte species. The polyelectrolytes can be any ionic solution capable of forming a layer on external and/or internal surfaces of the hydrogel scaffold and/or a previously coated polyelectrolyte layer, depending on the deposition or layering method. In non-limiting examples, the polyelectrolyte can be clay followed by poly(dimethyldiallylammonium) chloride (PDDA). Clay possesses a negative charge, and can therefore serves as a negative polyelectrolyte, while PDDA possesses a positive charge, and is a positive polyelectrolyte. In some embodiments, the polyelectrolyte can be any charged mixture or pure species, including without limitation, (PDDA), alumosilicate clay (montmorillonite), ionic polymers, for example, poly-lysine, oligonucleotides, poly acetylamine, collagen, alginate, carageenan, fibronectin, gelatin, extra-cellular matrix, poly(ethyleneimine) (PEI), poly(allylamine hydrochloride (PAH), poly aniline, polyacrylic acid, poly lactic acid, compositions containing cellulose, for example, cellulose nanocrystals, and carbon nanotubes.

In some embodiments, the ICC scaffold can be contacted with the polyelectrolyte in any manner commonly used in porous structure coating methodologies. For example, the ICC scaffold can be sprayed, dipped, washed or coated with the one or more polyelectrolytes or electrostatically attracted inside the scaffolds, using for instance electrocapillari phenomena or electrostatic attraction of the LBL component to external electrode. ICC scaffold itself may be made conductive by a producing a conductive coating on it, and thereby replace any additional electrode. For example, the ICC scaffold can be sprayed, dipped, washed with the one or more polyelectrolytes. In each dipping cycle, a (mono)layer of the species to be applied, adsorbs to the scaffold, while the rinse step removes their excess. The next dipping cycle results in enhanced adsorption of the oppositely charged species, which is also accompanied by a switch in the surface charge. This promotes the adsorption of the subsequent layer. Due to the monomolecular nature of the layers deposited in each cycle, the LBL technique affords nm scale precision in thin film thickness. This cycle can be repeated as many times as one need to build up a multilayer to a desirable thickness. The process can be easily automated and scaled-up.

Importantly, the assembled biopolymers retain their 3-D structure and biological activity.

In some embodiments, the ICC scaffolds can be coated with a variety of proteins from extracellular matrix (ECM), including without limitation, biopolymers such as collagen and fibronectin. It is contemplated, that the deposition of polyelectrolyte can improve the overall density of the cells seeded into and around the ICC scaffold.

The internal and/or external surfaces of ICC scaffolds can be coated with biologically functional molecules via LBL assembly. In some embodiments the ICC scaffolds can be coated in situations where there are large numbers of ICC scaffolds to be coated. In some embodiments, a different method can be applied to coat ICC scaffolds individually. In some embodiments, a coating method can be used to produce ICC scaffolds having surfaces that are biologically active and promote cell attachment, but are not specific to a cell type or function, such as directed differentiation or increased proliferation. In other embodiments, a second coating method can be used to produce ICC scaffolds coated with biomolecules intended for promoting attachment, growth, proliferation, or differentiation of a specific cell type. This second method is noted because as the intended function of the ICC scaffold becomes more specific, it may be more economical to utilize a method intended to produce smaller numbers of scaffolds.

### LBL Coating for Bulk ICC Scaffolds

In some embodiments, methods for coating large numbers of ICC scaffolds having surfaces that are biologically active and promote cell attachment comprise the step of placing all of the scaffolds into one or more polyelectrolyte solution sequentially. The general aim of LBL on the surface of a ICC scaffold is to promote cell attachment. In some embodiments, the LBL bulk-coated ICC scaffolds, are coated with the components chosen on the grounds of improved functionality and economic practicability.

Methods for LBL coating of bulk ICC scaffolds are described herein. First, ICC scaffolds can be placed in a bath of water to remove excess monomer. The bath can contain a built-in magnetic stirrer with adjustable stirring speeds, as well as a drain and water source to continuously replenish fresh water. In each step, the stirring bath serves to assist in diffusion of water or polyelectrolyte solution. The scaffolds can be placed into a rectangular metal net having the same dimensions as the bath. The scaffolds and net can be dipped into the bath vigorously stirring the scaffolds in the bath for approximately 30 minutes. Next, the scaffolds can be collected by removing the net from the bath and letting water drip from the net. The net containing the scaffolds can be transferred to a similar stirred bath of 0.5% PDDA solution for approximately 30 minutes. The scaffolds are then collected and transferred back to the water bath for about 15 minutes of rinsing, to rinse excess PDDA and ensure a monomolecular layer remains. The scaffolds are then collected and transferred to a 0.5% clay bath and stirred for another 30 minutes. After coating with PDDA, rinsing with water, coating with clay, and rinsing with water, the scaffolds are considered to have received one layer. In an exemplary embodiment, the coating and washing steps can be repeated at least five times so that five layers can be coated on the scaffold surface. After the fifth layer is applied, the scaffolds are replaced in a water bath for storage. Since the number of layers to be coated on the ICC scaffold can vary, the number of steps can vary accordingly. Similarly, the duration of the coating and washing steps can easily be adjusted according to the coating's composition and its intrinsic capability to adhere to the layer applied before it. In lieu of clay one can also use other colloidal materials, for example nanocolloids of cellulose in different varieties, dispersions of extracellular matrix, proteins, carbon nanotubes, nanoparticles, and other adhesion promoting materials. Also, the scaffolds can be coated with nanometer scale layer(s) of biocompatible materials facilitation specific cellular response by reaction in the bulk of the fluid infiltrating the scaffold. These type of coatings can be applied directly onto the polymer, for example a hydrogel, or on LBL layers serving as a substrate for subsequent coating of the scaffolds. In some embodiments, coatings comprising SiO₂ by controlled hydrolysis of its precursors or by calcium phosphate by precipitation reaction of two salts. Both coatings are expected to enhance cellular adhesion. One of ordinary skill can unduly experiment and obtain optimal incubation times for the particular coating required.

### LBL Coating for Individual ICC Scaffolds

In embodiments requiring the application of layers of polyelectrolytes to small numbers scaffolds or individual ICC scaffolds, polyelectrolyte coating is not done in large containers. In some embodiments, ICC scaffolds to be coated in this smaller scale process, can be treated with clay/PDDA as described above, to impart the benefit of increased cell attachment as well as greater biomolecular activity. To treat and coat fewer scaffolds, in accordance with the present embodiment, an ultrasonic bath can be used to assist diffusion, rather than a stirring bath. In some embodiments, the polyelectrolyte and/or washing solutions can be dispensed manually using for example a pipette or other similar apparatus, or the solutions can be dispensed automatically. In some embodiments, the automated microplate pipetting system can be used to dispense the polyelectrolyte and/or washing solutions. First, ICC scaffolds can be placed into one or more wells of cell culture well plates (having a number of wells ranging from 1-1536). The automated microplate pipetting system can introduce the first polyelectrolyte solution ranging from several mL to several microliters into the well or wells of the cell culture plate. Gentle sonication can be applied for 15 minutes to facilitate diffusion of the coating materials into the 3-D ICC scaffolds, while at the same time prevent damage to the formerly coated film. Next, the polyelectrolyte solution can be removed from the well, and deionized water can be added into the well and gently sonicated for about 30 minutes to remove excess polyelectrolyte.

After removing the wash water, a solution of polyelectrolyte or a bioactive agent, can be added to the ICC scaffolds. Note that the polyelectrolyte can be chosen to possess an opposite charge to that of the desired bioactive agent. Lastly, the bioactive agent solution can be removed and water can be added to the scaffolds to rinse the excess bioactive agent. This process generally requires only 1-5 applications of polyelectrolyte/bioactive agent to achieve surface activity.

In some embodiments, one or more bioactive agents can be added to the hydrogen ICC scaffold to render the scaffold biocompatible and/or tissue selective, i.e. possessing the required biological molecules which can influence an attached cell to grow, perform a cell function such as differentiation or activate or repress a signal. In some embodiments, the ICC scaffold can further contain one or more added bioactive agent, either: (1) encapsulated in one or more hollow space(s) within a “hollow” void; or (2) located within or throughout the bulk of a “solid” particle, or of a core, wall, or layer of a hollow or laminar particle, or surface or wall of a void and/or pore. Examples of bioactive agents for use in an embodiment of the present teachings are: bone morphogenic proteins (e.g., BMP1-BMP15), bone-derived growth factors (e.g., BDGF-1, BDGF-2), transforming growth factors (e.g., TGF-α, TGF-β), somatomedins (e.g., IGF-1, IGF-2), platelet-derived growth factors (e.g., PDGF-A, PDGF-B), fibroblast growth factors (e.g., αFGF, βFGF), osteoblast stimulating factors (e.g., OSF-1, OSF-2), and sonic hedgehog protein (SHH); notch protein, other hormones, growth factors, and differentiation factors (e.g., somatotropin, epidermal growth factor, vascular-endothelial growth factor; osteopontin, bone sialoprotein, a 2HS-glycoprotein; parathyroidhormone-related protein, cementum-derived growth factor); biogenic proteins and tissue preparations (e.g., collagen, carbohydrates, cartilage); gene therapy agents, including naked or carrier-associated nucleic acids (e.g., single- or multi-gene constructs either alone or attached to further moieties, such as constructs contained within a plasmid, viral, or other vector), examples of which include nucleic acids encoding bone-growth-promoting polypeptides or their precursors, e.g., sonic hedgehog protein (see, e.g., P C Edwards et al., Gene Ther. 12:75-86 (2005)), BMPs (see, e.g., C A Dunn et al., Molec. Ther. 11(2):294-99 (2005)), peptide hormones, or anti-sense nucleic acids and nucleic acid analogs, e.g., for inhibiting expression of bone-degradation-promoting factors; pharmaceuticals, e.g., medicaments, anti-microbial agents, antibiotics, antiviral agents, microbistatic or virustatic agents, anti-tumor agents, and immunomodulators; and metabolism-enhancing factors, e.g., amino acids, non-hormone peptides, toxins, ligands, vitamins, minerals, and natural extracts (e.g., botanical extracts). The bioactive agent preparation can itself contain a minority of, e.g., processing, preserving, or hydration enhancing agents. Such bioactive agents or bioactive agent preparations can be contacted into and onto the ICC scaffold through any dispensing means, for example, diffused, sprayed, suctioned, imbibed or added to the polymer solution directly before forming the three dimensional matrix, or both. Where both the void and pore surface and polymer solution contain bioactive agent(s), the agent(s) can be the same or different. It should be appreciated however, that the present disclosure is not limited by any particular method of treating the ICC scaffold with a bioactive molecule, for example a growth factor, and the disclosure is applicable to any such method now known or subsequently discovered or developed.

In some embodiments, growth and/or differentiation factors useful in the present disclosure can include, but are not limited to: sonic hedgehog, notch ligand, vascular endothelial growth factor (VEGF), epidermal growth factor (EGF), fibroblast growth factor (FGF), insulin growth factor (IGF), erythropoietin (EPO), hematopoietic cell growth factor (HCGF), platelet-derived growth factor (PDGF), nerve growth factor (NGF), transforming growth factors a and β (TGF-α and TGF-β), bone morphogenetic protein 1-17 (BMP 1-17) or combinations thereof.

### Incorporation of the Biologically Active Agents in the 3-D ICC Scaffold.

Biologically active agents can be applied to the hydrogel matrix of the scaffolds before or after placement in the well-plates. Scaffolds can be contacted with a desired chemical or biological active material, to be incorporated in the wall, cavities and pores of the 3-D hydrogel matrix using for example, vacuum suction, spraying, immersing in a bath or wetting techniques. After a desired incubation period, the chemical or biological active material can be removed and scaffolds will retain a specific amount of the chemical or biologically active material due to entrapment/adsorption in/on the hydrogel matrix.

### Transfer to Cell Culture Well Microplate and Packaging

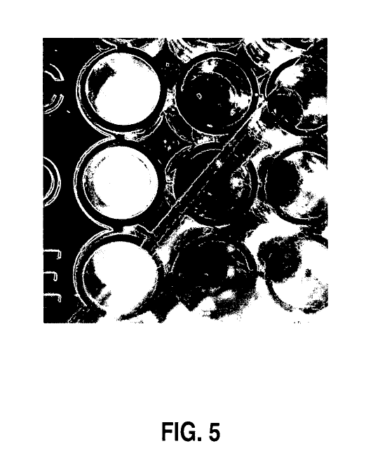

In some embodiments, the present disclosure includes two methods of packaging the ICC scaffolds. In some embodiments, a method for storing hydrated ICC scaffolds can be employed. The final ICC scaffold samples can be placed in one or more cell culture well microplates with a compatible sterile solution, for example, deionized water or phosphate buffered saline solution. The cell culture well-plate can then be covered by a sealing tape as shown in FIG. 5. After the cell-culture well plate is sealed the scaffolds can be sterilized using any compatible and convenient means, for example, sterilization under UV or radiation, gamma-radiation, electron beam or the like for up to 12 hours, so that the scaffolds are ready-to-use. In some embodiments, the sealed scaffolds can be sterilized chemically, for example with ethylene oxide and chloride dioxide. The ready-to-use sterilized hydrogel scaffolds can be stored in a 4° C. refrigerator prior to delivery and use.

The second method is to pack ICC scaffolds after a dehydration process, for instance freeze-drying also known as lyophilization. In some embodiments, ICC scaffold samples are immersed in liquid nitrogen for 5 min, and then placed and lyophilized in a freeze drying machine for about 12 to about 24 hours. This process minimizes the shrinkage of ICC scaffolds, curtailing damage of coated materials. Dehydrated ICC scaffolds can be temporarily glued in a cell culture well-plate utilizing 50:50 poly(lactic-co-glycolic acid) (PLGA) polymer. In some embodiments, the cell culture plate can also be covered by a sealing tape. The ICC scaffolds can be sterilized using a chemical gas or sterilized under UV radiation for approximately 12 hours and stored in a room temperature desiccator. Dehydrated scaffolds can easily intake deionized water or phosphate buffered saline solution within one hour, and thereby recovering its original biological and physical properties. Because the scaffolds can be glued with PLGA, the scaffolds can be stationary during the re-hydration process. The second desiccation method can be tailored for long-term storage of ICC scaffold samples.

### Methods of Use

ICC scaffolds are ideally suited as a 3-D cell culture substrate because of its highly porous and mechanically stable structure. The highly ordered and uniformly sized porous geometry can be replicated with great consistency, and can be made adjustable by altering the microsphere size and annealing temperature which can control the size of the cavities and interconnecting pores. In some embodiments, the internal and external surfaces of a ICC scaffold can be coated with various biological molecules utilizing a layer-by-layer (LBL) molecular assembly technique that can be used for coating oppositely charged polyelectrolytes. A large variety of biomolecules can be stably deposited and applied to the surfaces of the pores and of the surfaces of the three dimensional matrix through the LBL method with minimal loss of bioactivity. Low mass transport resistance within the ICC structure permits a uniform coating and ultra-thin multilayers on the complex 3-D porous substrate with nanometer precision. As a result, LBL-coated ICC scaffolds have precisely designed micro- and and nano-scale geometry and surface properties. Due to its simple and robust fabrication procedure, a consistent 3-D microenvironment can be maintained.

### Preparation of ICC Cell Scaffolds

In some embodiments, the ICC scaffolds described herein can be used to selectively grow and culture living cells. As used herein, living cells can include bacteria, algae, yeast, plant cells and animal cells. In some embodiments, the living cells can be selected from the group consisting of myocyte precursor cells, cardiac myocytes, skeletal myocytes, satellite cells, fibroblasts, cardiac fibroblasts, hepatocytes, chondrocytes, osteoblasts, removal cells, endothelial cells, epithelial cells, embryonic stem cells, hematopoetic stem cells, neuronal stem cells, hair follicle stem cells, mesenchymal stem cells, and combinations thereof. Preferably, the living cells are mammalian cells including for example, rabbit, dog, goat, horse, mouse, rat, guinea pig, monkey, and human cells. Still preferably, the mammalian cells are human cells. In some embodiments, ICC scaffolds can be prepared as described above and can be tailored for the growth or many different types of living cells, the growth of specific types of cells, or enable one or more cell types to become differentiated into a different lineage of cells.

In some embodiments, the ICC scaffolds can be seeded with living cells using any commonly known cell seeding technique, including without limitation, a liquid dispensing means to aseptically transfer cells from one container to the well containing the ICC scaffold, for example, a pipette, spraying a cell culture onto and into a ICC scaffold, filtering a cell culture through a ICC scaffold and by centrifuging a cell culture solution on top of a ICC scaffold and combinations thereof.

In some embodiments, the cell culture plates can contain identical ICC scaffolds having identical matrix coatings but different cell culture conditions, for example, different culture media. Alternatively, the cell culture plates can contain identical ICC scaffolds having different matrix coatings but identical cell culture conditions, for example, each well having a different biological molecule adhered to the polymer matrix during the LBL process. In these embodiments, each well can contain the same media. The result of analysis of cell behavior in each well will allow the experimentalist to choose optimal conditions for specific biological system or a specific cell type.

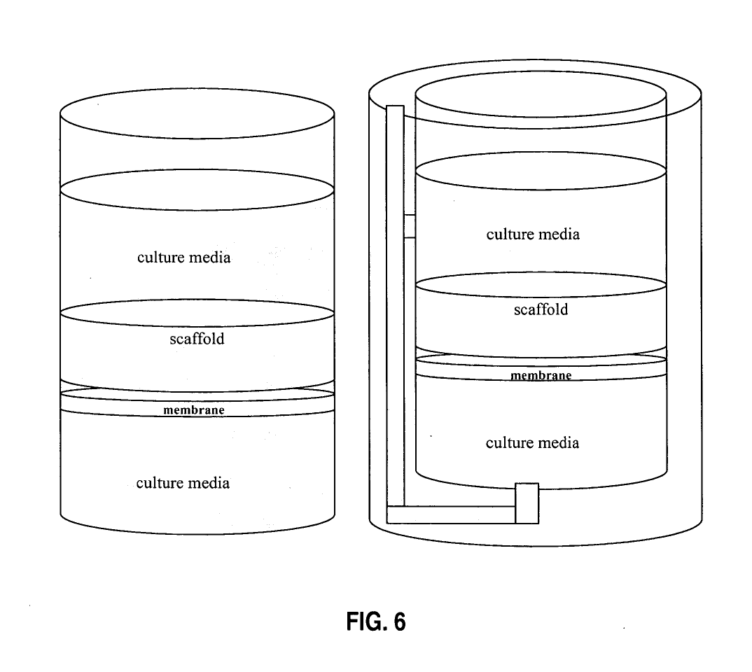

In some embodiments, cells can be cultured in ICC scaffolds further comprising a recirculating media system. The cell culture plates may also have specially engineered channels and/or supply mechanisms that can facilitate the delivery of nutrients to all the parts of the scaffold and especially to the bottom of the scaffold. An ICC having a recirculating media system is shown in FIG. 6.

### Maintenance and Expansion of Stem Cells

In some embodiments, implementation of cell culture microplates with ICC scaffolds for selecting stem cell conditions for proliferation and differentiation can be made. In some embodiments, the ICC scaffold can be layered with one or more desired biological molecules, including growth factors and receptor ligands, and seeded with one or more stem cells, including embryonic stem cells, hematopoetic stem cells, neuronal stem cells, mesenchymal stem cells, and hair follicle stem cells. In some embodiments, the cell culture media in wells will be varied and the reaction of stem cells on the presence or absence of specific components in the ICC scaffold or tissue culture media can be analyzed. According to these results, the choice for specific biological molecules, including growth factors and different media components for optimal stem development and/or expansion can be made.

Approaches to the in vitro expansion of stem cells, for example, without limitation, embryonic stem cells, hematopoetic stem cells, neuronal stem cells, mesenchymal stem cells, and hair follicle stem cells generally involve techniques that utilize stromal cell support, growth and differentiation factors and/or addition of cytokines. In another embodiment of the present disclosure, expansion of the hematopoetic stem cell (HSC) population without induction of maturation or differentiation of the cells can be accomplished by culturing the HSCs in the presence of bone marrow stromal cell HS-5 seeded on the ICC scaffolds within a rotary cell culture system bioreactor.

In some embodiments of the present disclosure, 3-D ICC scaffolds can be particularly useful can be the cellular assays for the development of different vaccines. CD34+ stem cells can serve as precursors to a number of hematopoietic cells including B-cells developing in the bone marrow. Differentiation of HSCs into pro-B cells and finally into pre-B cells is a stepwise progression that requires sequential expression of lymphoid regulatory genes as well as somatic rearrangements of the immunoglobulin heavy and then light chain genes. Rearrangements of the light chain genes are followed in immature B cells by the expression of cell surface IgM. Mature B cells express both IgM and IgD on their surface and it is at this stage that the mature but antigen-naive B cell exits the bone marrow and enters the peripheral circulation.

In this case, a cellular culture with a subset of immune system cells or HSCs can be cultured in the wells containing a ICC scaffold. The addition of pro-B-cell lineage growth and differentiation factors can elicit a phenotypic differentiation into one or more B-cell subsets. In some embodiments, exposure of one or more specific antigens administered either in the culture media, or tethered to the ICC scaffold seeded with a mixed B-cell population can be used to gather knowledge of the antigen-immune response. A greater understanding between the cells of the immune system and a particular antigen can permit rational design of antigen structures for vaccine development. The analysis of this reaction will enable optimization of the vaccine composition and methods of its preparation.

### Drug and Pharmaceutical Testing and Evaluation

In addition to various antigen preparations, pre-existing and candidate compounds can be tested for biological activity or toxicity using in vitro and in vivo constructs employing ICC scaffolds seeded with cells. Designed drug candidates with individual chemical structures, as well as various drug formulations, such as vaccines, anticancer drugs, antiviral drugs and others, can be tested initially on cell cultures in order to maximize potential curing effects and evaluate the potential toxicity, prior to animal and human trials. The overall research and development cycle for drugs costs $300-800 million in capital and up to 10-12 years in time. One of the reasons for such great cost is that the vast majority of drug candidates are screened out at the stages of animal and human trials. More efficient methods of testing of drugs at any stage of drug development, particularly at the stage of ex-vivo studies, which are substantially less expensive than animal and human testing cycles, will lead to acceleration of drug discovery, reduction of the cost of pharmaceutical development, and better drugs. This particularly true for advanced drugs for HIV, cancers, metabolic, immunological and autoimmune deceases. Efficacy of in vitro testing can be significantly improved provided that better ex-vivo models for different organs and tissues are developed. A large body of research indicates that cultured cells organized in three-dimensions (3-D) behave a lot more closely to the original tissues and retain more natural functions than the cells in 2D cultures. Driven by the desire of the pharmaceutical industry to replace some portion of regular in vitro drug testing and potentially some animal trials with experiments in 3-D cell cultures, which can reduce the cost and the time from inception to production of a new drug, much work is also being done on the development of replicas of key body tissues.

A large variety of materials and manufacturing processes have been used to make 3-D scaffolds for previous 3-D in vitro drug testing studies. However, many of them are not convenient for mass drug testing protocols due to strong light scattering/absorption and variability in the scaffold quality/topology. Virtually all of the commercially made 3-D scaffolds are made from ceramics or other inorganic materials and are difficult to incorporate in the developed drug evaluation protocols. Several advantages can be afforded to transparent ICC scaffolds, and can include ease of monitoring and examining changes in cell function after administration of a compound for example, a toxin, a pharmaceutical, a drug, or a candidate compound to the cell.

In some embodiments of the present disclosure, the present disclosure further contemplates a method of identifying the effects of a toxin, a drug, a medicament or a pharmaceutical composition, or an infectious agent for example a bacterium or viral pathogen on cell function comprising administering a compound in vitro to an inverted colloidal crystal scaffold seeded with viable cells; and determining the affects of the compound on the living cells by measuring, collecting, or recording information on the cells or products produced by the cells. In some embodiments, the cells to be tested can be any mammalian cell including without limitation, myocyte precursor cells, cardiac myocytes, skeletal myocytes, satellite cells, fibroblasts, cardiac fibroblasts, hepatocytes, chondrocytes, osteoblasts, endothelial cells, epithelial cells, embryonic stem cells, hematopoetic stem cells, neural cells, neuronal cells, hair follicle stem cells, mesenchymal stem cells, and combinations thereof. In some embodiments, the cells to be studied include bone marrow cells, cardiac myocytes, hepatocytes and neural cells.

In some embodiments, the method further comprises determining the effects of a compound on the cells by measuring or identifying changes in cell function. This can be accomplished by many methodologies known to those skilled in the art including, for example, Western blot analysis, Northern blot analysis, RT-PCR, immunocytochemical analysis, flow cytometry, immunofluorescence, BrdU labeling, TUNEL assay, and assays of enzymatic activity assays of enzymatic activity, and automated assays encompassing the above measurement assays in a highly sequential manner high throughput and high content analysis, for example nucleic or protein chip arrays using fluorescently labeled molecules.

In some embodiments, the living cells are hepatocytes. Accordingly, measurements of parameters such as albumin production and liver enzyme activity can be made. By way of example, the instant ICC scaffold containing hepatocytes clustered into spheroids that can be cultured indefinitely in the ICC scaffold could be treated with one or more agents or compounds, such as a liver toxin, statins, cholesterol, or lipoprotein, and any change in the production of albumin, cholesterol, detoxifying enzymes, and other liver enzymes can be measured as described above. This method provides an in vitro diagnostic system that can be utilized to rapidly assay the physiological consequences of administration of a given drug, pharmaceutical composition, medicament or toxin on cell function such as, production of albumin and liver enzymes.

In some embodiments, testing of the drugs affecting the brain and Central Nervous System can be modeled in vitro using ICC scaffolds cultured with the appropriate target cells or tissue. In some embodiments, the target tissue or cells are neural tissue or neural cells. Currently experimental 3-D models for neural tissues are not available. 2-D neural cell cultures lack exceptionally important connectivity components along the cells, which are targeted by many drugs. Also the ICC scaffolds of the present disclosure are contemplated to be exceptionally helpful in the understanding cellular interactions between different cells in neural tissue, such as the cellular interactions between neurons, oligodendrocytes, glial cells, Schwann cells and the like particularly useful to study the pathological processes in neurological diseases, for example as in Alzheimer disease and Parkinson's disease.

In some embodiments, a diagnostic assay or system can be realized comprising administering a pathogen or infectious agent, for example with a bacterium or virus, to a cultured cell or cultured cells, for example liver cells infected with hepatitis virus or T-cells infected with HIV Production of artificial tissue. In some embodiments, the ICC scaffolds can be manipulated to provide scaffolds enhanced for cellular infiltration, integration and remodeling of introduced cells. In some embodiments, the ICC scaffolds of the present disclosure can be utilized to grow any living cell as enumerated and described above. The ICC scaffolds of the present disclosure can be made to grow and reconstruct living tissue material. In some embodiments, the tissue can be artificial skin, hair follicles, blood vessels, bone marrow, neural tissue, muscle, cardiac muscle, liver tissue, bone and cartilage. Cells used for reconstruction of autogenous or allogeneic tissue can be of any type typically residing in the tissue type to constructed. In some embodiments, stem cells can be used to provide the progenitor source of cells to be grown in vitro. Stem cells are ideally suited for the construction of autogenous and allogeneic tissue because they can be readily isolated form the patient, for example, mesenchymal stem cells from bone marrow, skin stem cells from the dermis, adipose derived stem cells from lipectomy procedures (liposuction), hair follicle stem cells from hair transplants. In some embodiments, embryonic stem cells from mammalian sources, including for example human embryonic stem cells can be used to create any tissue type in an artificial construct using ICC scaffolds. For the purpose of regeneration of tissues ICC scaffolds can be made from biodegradable materials such as PLA, PLGA, hyaluronic acid, collagen, etc.

Various stem cell growth and differentiation factors are relatively well known and have been successfully used to produce differentiated cells from progenitor and stem cells in vivo and in vitro. It is contemplated by the teachings of the present disclosure, that artificial tissue can be used to graft and repair tissue due to disease and trauma, to replace tissue to correct a congenital aberration and to enhance and augment cosmetic procedures.

### Possible Variations and Modifications

The dimensions of the ICC scaffold can be modified to fit microplates with different sized wells. For example, the vial size used to make colloidal crystals can be altered to fit the dimensions of 24-, 48-, 96-, 384, or 1536 well microplates, and ICC scaffolds fitting these microplates can be designed. This may be beneficial for studies, such as such as tissue engineering, requiring greater numbers of cells or longer cultures than allowed by a cell culture well microplate. Additionally, the dimensions can be modified to fit a perfusion bioreactor. This is particularly useful when not only are cells a final product, but also if a molecule produced by cells, such as an antibody or a hormone, is the desired product.

The materials of which ICC scaffolds are made can be changed according to a specific application. For example, a biodegradable polymer, such as PLGA or poly(e-caprolactone) can be substituted for hydrogel. Any material that is soluble in liquid, where consistently-sized microspheres that will not be dissolved by the same solvent exist, can be used to create an ICC scaffold. Also, it was mentioned in the technical description of the LBL process that the LBL coating materials can be altered.

Additionally, the present disclosure is also directed to a commercial kit comprising inverted crystal colloidal scaffolds sealed in a sterile package and instructions for use thereof in culturing cells. The scaffold can be included in a kit that includes a sterile polystyrene tissue culture plate with the standard number of wells 6, 12, 24, 48, 96 384 or 1536 wells within which the scaffolds have been placed, instructions for the cellular seeding and/or optimal dispersion concentration of growth/active factors, and accessory tools for proper scaffold handling. In a different approach, the present disclosure can feature a kit that includes sterile pre-formed three-dimensional scaffold shapes, a lyophilized or a combination of lyophilized growth/active factor(s), associated tools to allow the delivery of the lyophilized agents homogenously within the scaffold, and instructions for proper growth/active factor dispersion. In a different approach, the present disclosure can feature a kit that includes sterile pre-formed 3-D scaffold shapes, a lyophilized or a combination of lyophilized growth/active factor(s), a photopolymerizable agent, a vial to mix the photopolymerizable agent with the lyophilized compound, associated tools to allow the homogenous distribution of the photopolymerizable agent plus lyophilized compound into the scaffold, and necessary instructions. The kit could or could not include a light source to induce local photopolymerization, thus, trapping of the lyophilized compound into the 3-D scaffold.

The following examples describe embodiments within the scope of the claims herein, and other embodiments within the scope of the claims will be apparent to those skilled in the art from an understanding of the specification or practice of the disclosure as disclosed herein. It is intended that the specification along with the examples are to be considered as exemplary only, with the scope and spirit of the present teachings being indicated by the claims. In the examples, all percentages are given on a weight basis unless otherwise indicated.

### Examples

### Example 1

Construction of ICC Scaffolds for Tissue Growth and Repair

ICC scaffolds can be made with poly(lactic-co-glycolic acid) (PLA-PLGA) that has a lactic to glycolic acid ratio of 85:15. The co-polymerized polymer has a faster degradation rate than each of the single components, i.e. PLA or PLGA. They are very stable at room temperature when stored in dry format.

An upside-down beaker was placed into a sonication bath, and a 9 mm outer diameter vial is clamped on top of the beaker. A 9 in Pasteur pipette is clamped with its narrow end suspended inside the center of the vial. Teflon tape can be used to seal the opening of the vial and secure the pipette in the center, and the apparatus was filled with ethylene glycol. Soda lime spheres (Duke Scientific, Palo Alto, Calif.) with a diameter of 99.8 μm and size distribution of 3.2% are added to ethylene glycol in a dropping bottle. Under constant sonication, two drops of spheres are dropped through the pipette into the vial every fifteen minutes until the precipitated spheres has reached a desired height. The pipette is removed, and ethylene glycol is evaporated from the vial at 160 ° C. overnight. Spheres can be then heated at 675 ° C. for 3 h to anneal adjacent spheres into a solid colloidal crystal. The colloidal crystal is then infiltrated by submerging in 10% (w/v) 85:15 polylactic-co-glycolic acid) (Absorbable Polymers International, Pelham, Ala.) in dichloromethane, and sonicated for 3 hours or centrifuging at 5,900 rpm for 10 minutes. Infiltrated colloidal crystals are then placed into a vial with a small volume (to cover colloidal crystals) of 10% PLGA, and solvent is allowed to evaporate at room temperature overnight and under vacuum for an additional 24 hours. Soda lime beads can be removed from the composite colloidal crystal scaffold by stirring in 1% HF for 3 h, followed by rinsing several times with water. The resulting inverted colloidal crystal structure can then be examined by light microscopy for complete removal of soda lime beads. If beads are visible on the surface, a layer of PLGA can be scraped off the surface with a razor, and re-immersed in HF. This can repeated until all beads are removed.

PLA-PLGA has been known as a material for regenerative medicine, but in case of artificial skin, grafts with greater flexibility and ability to conform to the body curvatures are desired. Alginate can be used to make such scaffolds. Alginate is a biodegradable scaffolding material with the mechanical properties similar to that of hydrogel. The calcium alginate scaffolds can be prepared from high-G alginate and calcium chloride by the gelatin-freeze technique,ⁱ which consists of the following steps: (1) preparation of 2% (w/v) sodium alginate stock solutions; (2) cross linking the alginate solution by adding an equal volume of calcium chloride solution (the final concentration of Ca²⁺ is 0.01 M), while stirring intensively using a homogenizer at 2,000 rpm for min; (3) transferring the sol-gel into a dish or into the colloidal crystal mold and freezing the cross-linked material, at −18° C., overnight; and (4) melting the frozen material at room temperature. After the removal of the microspheres, the resulting gel like sponges are cut into small pieces and can be sterilized using ethanol solution and stored in distilled water at 4° C. until use.

LBL coating on PLGA scaffolds with collagen. To construct an epidermal supporting layer on the ICC scaffold, collagen can be coated onto the PLGA ICC scaffold via LBL assembly. Polyacrylic acid (PAA) polyelectrolyte is used as a counterpart for the LBL deposition. Due to the negatively charged nature of PLGA, the coating can be applied by alternate deposition of positively charged collagen (type I from calf skin, 0.2 mg/mL in 0.1 N acetic acid solution, pH=4) and oppositely charged PAA (1 mg/mL, pH=3) onto the scaffold.

A Microlab STAR liquid handling system (USA) can be used to apply the coating automatically. The scaffold can first be placed into a well of microplate (48 well). 400 μL collagen solution is transferred into the well with a pipette programmed automatically and kept for 20 min for the deposition of collagen layer on scaffold. After the collagen deposition, the collagen solution is removed from the well for disposal. De-ionized (DI) water is then brought into the scaffold well to rinse the scaffold twice for 5 min (2.5 min each). Following the same procedure, 400 μL PAA can be transferred into the well, and the solution is left for 10 min for the PAA layer deposition, followed by D.I. water rinsing twice for 5 min after PAA solution is removed. The same cycle can be repeated until the desired layer numbers was obtained. In our preparation, the LBL coating can be carried out repetitively to achieve 37 bilayers of collagen/PAA [(collagen/PAA)₃₇] on PLGA scaffold. In order to estimate the coating layer thickness, structure and topography, the first and last layers of PAA are replaced by fluorescent-labeled trypsin inhibitor (10 μg/mL) which emits in green channel for confocal observation. The scaffold can be sectioned in order to inspect the cross section for the LBL coating. UV-Vis spectroscopy, transmission optical microscopy, confocal microscopy, atomic force microscopy, and SEM can be used to inspect the coated scaffold.

Degradation rate of PLGA ICC scaffold. The PLGA scaffold size can shrink about 25% over 2 weeks in PBS buffer (pH 7.4). This rate can be used for assessment of the degradation of the prepared scaffolds in-vivo, although we observed that the decay of ICC constructs in mice is significantly faster than in PBS buffer. The rate of scaffold can shrink slowed down after the first two weeks, which is possibly due to the degradation kinetics following an exponential decay pattern. This can be controlled by optimizing the architecture of the scaffold.

Biocompatibility of the ICC-LBL hybrid scaffolds. The biocompatibility of the ICC-LBL hybrid scaffolds with in vitro cell cultures can be tested using ICC scaffolds having a functionalized matrix including voids surfaces and pores. The hybrid scaffolds can be pre-soaked with culture medium (DMEM with 20% FBS and 1% Pen-Strip) for 24 hours. Three mouse cell lines: epithelial XB-2, endothelial MS1 and fibroblast STO, can be seeded on the hybrid scaffolds that are placed in wells of a 48-well microplate, the number of cells per scaffold was 5×10⁴ for each line. Culture media can be changed every 48 hours. One week later the cells can be stained with a fluorescence viability kit commercially available from Molecular Probes Inc. (Eugene, Oreg. USA) and can be inspected with a confocal microscope.

### Example 2

Development of the Bone Marrow Construct. A bone marrow construct would have to support the self renewal of an undifferentiated population of CD34+ HSCs for a period of at least 4 weeks and the construct would also have to support the production of fully functional immune cells of a specific leukocyte lineage. In order to produce a bone marrow construct, stromal or feeder cells can be seeded onto ICC scaffolds and cultured for 3 days to allow formation of a dense layer on the scaffold surface or in plate cultures prior to the addition of CD34+ HSCs. CD34 HSCs can be used since they have been shown to provide for long term multilineage engraftment capability. CD34+ HSCs from a variety of sources can be selected to evaluate the capacity of each of these populations of early HSC progenitors to replicate the functions of natural bone marrow. CD34+ HSCs can be isolated from human peripheral blood, umbilical cord blood or bone marrow using counter current centrifugal cell elutriation followed by flow cytometric cell sorting to remove any lineage-1 (Lin-1) positive or mature cell types. All cells positive for CD34 can be seeded onto the scaffolds and in all samples a small portion (1-2%) can be low CD34 expressing cells also positive for CD150, a cell marker associated with long term multi-cell lineage reconstitution in irradiated mice.

An examination of ICC cultures on day 14 can show a continued presence of CD34+ HSCs. Numerous actin-rich cell processes can be seen in ICC matrixes but not in plate cell cultures after 28 days of culture. A population of CD150+ cells can be seen in 3-D ICC/HSC cultures but not in donor matched 2D cultures after 28 days. There can be significantly higher percentages of CD34 expressing cells in 3-D ICC cultures after 28 days, regardless of the cell source, when compared to donor matched 2D plate cultures. Large reductions of CD34+CMFDA staining indicative of cell proliferation can be seen by CD34+ cells in 3-D ICC cultures but only low levels may be seen in donor matched 2D plate culture proving for the time periods evaluated that there can be some expansion of the original CD34+ cell population seeded into the cultures and that an undifferentiated population of CD34+ cells can be maintained over time.

### Example 3

Stem Cell Differentiation. To assess the ability of the artificial bone marrow constructs to produce fully functional immune cells B lymphocyte production can be used specifically, since B cells normally undergo the process of differentiation (as well as negative and positive selection) in the bone marrow. B cell development involves a series of stages where close 3-D contact between bone marrow stroma and the developing B cell is critical and is hard to realize in plate cultures. Bone marrow is not only involved in B-cell differentiation but it is the site of long term antibody production after viral infection and bone marrow stroma has been shown to play a role in plasma cell longevity. After 3 days of culture, ICC/stromal cell constructs can be seeded with CD34+ HSCs. Cell cultures can be examined for stage specific markers of development on days 1, 7, 14, 28 and 40 of culture. Nuclear specific expression of Rag-1 by day 7 can be identified in ICC scaffolds, cell surface IgM by day 14 and by day 28 co-expression of IgM with IgD may be seen, confirming mature antigen naive B lymphocyte generation. In our experiments, cell surface expression of CD40, IgM, IgD, and coexpressed IgM and IgD can be evaluated on day 28 for both 2D and 3-D cultures. Significantly higher levels of CD40 and coexpressed IgM and IgD can be seen in donor-matched 3-D compared to 2D cultures. Expression of phenotypic cell surface markers of differentiation does not necessarily prove the functionality of the ex-vivo generated B lymphocytes. To evaluate the ability of these of these B lymphocytes to respond to mitogenic or antigenic stimulation and fully mature into antibody producing cells, B lymphocytes isolated from 28 day ICC scaffold constructs and donor matched plate cultures can be exposed to bacterial lipopolysaccharide (LPS). Significantly higher levels of IgM can be produced from B lymphocytes generated in the 3-D ICC scaffold regardless of the initial source of the CD34+ cells.

In a subset of experiments artificial bone marrow constructs can be prepared as described above. Hydrogel ICC constructs can be seeded with human cord blood derived CD34+ HSCs. Cultures can be primed to proliferate and the B cell population can be expanded using anti-IgM crosslinking on day 14 of culture. Cultures can then be exposed to heat killed whole influenza A virus (MOI of 10) on days 28-30 of culture. Cultures yielding mature, IgG expressing cells after day 40 of culture as analyzed by confocal microscopy or flow cytometry can be shown. These IgG expressing cells, with an average production of 13.5+/−9.4% IgM expressing (with no expression of IgD) and 3.1+/−1.9% IgG expressing cells can be experimentally found. Examination of influenza A antigen specific antibody production by ELISA, hemagglutinin inhibition assay or virus neutralization assay can show low levels of consistent production of specific antibody in all ICC scaffold cultures but never in donor matched plate cultures receiving the same treatments. Low but consistent production of anti-IgG antibody for influenza HA can be shown.

3-D ICC scaffold/stromal cell constructs seeded with CMFDA labeled cord blood derived CD34+ HSCs and cultured in vitro for 7 days can be implanted on the backs of two SCID mice as a proof of concept test of in vivo functionality. The animals can be sacrificed after 2 weeks and then the implanted matrix, mouse bone marrow and spleens can be collected for leukocyte subset phentotyping. In various replicate experiments it can be shown that the majority of cells in the ICC scaffold two weeks after implantation can be CMFDA+ human MHC class I+ and that subsets of HSCs including CD34+, CD150+, and CD13+ can be maintained. Flow cytometric evaluation of cells isolated from the bone marrow of these mice can show that 75% of the cells can be human CMFDA+ MHC class I+ and that +91% of the CFFDA+ CD34+ cells originally implanted can undergo at least or more rounds of cell division.

Confocal analysis of cytospins of the murine bone marrow cells can demonstrate that CMFDA+ human CD34+ cells can engraft into the mouse bone marrow and that CMFDA+ CD10+ and CD7+ precursors to human T and B cells can also be found. Examination of the cells populating the spleens of these mice can indicate that the majority of cells (89%) can be of human origin as indicated by CMFDA staining and evaluation of MHC class I co-staining and that the predominant cell type can be CD19+ and IgD+IgM positive. Both CD133+ and CD150+ positive cells can be present in the reconstituted mice. Both of these populations have been previously shown to function in the repopulation of HSCs.

Mouse embryonic stem cell growth and differentiation can be analyzed using refined 3-D ICC scaffolds, co-cultured with selected skin cell lines (such as epithelial XB2, endothelial MS1 and fibroblast STO). The C57BL6 strain of murine ESCs can be divided into equal aliquots of 0.5-1×10⁵ cells and can be seeded into ICC scaffolds of different geometries made previously and that will be placed in a multi-well microplate. Appropriate combinations of growth factors can be added in order to induce the cells to differentiate towards a smooth muscle, neural, chondrogenic or adipose lineage. For the differentiation of cells to a smooth muscle lineage: DMEM/F12 w/10% fetal calf serum, and 3% human serum will be added. For differentiation to a neural lineage: DMEM/F12 w/10% FBS, 10% fetal calf serum, and b-mercaptoethanol (13-ME) can be added. If necessary, for development of the chondrogenic lineage serum free DMEM/F12+ITS+premix and TGF-β1 can be used. For the development of an lineage, DMEM w/10% fetal calf serum, Dexamethasone and indomethacin can be added to the culture. The cells will be allowed to incubate after nine days of culture at 37° C. in 5% CO₂. The wells will be evaluated for development and expression of lineage specific differentiation markers. Growth factors used to produce multiple cell lineages are as follows:

Adipogenic Medium: DMEM w/10% fetal calf serum, 50 μg/ml ascorbate-2 phosphate, 10⁻⁷M dexamethasome, 50 μg/ml indomethacin.

Chondrogenic Medium: serum free DME/F12+ITS+premix, 10 ng/ml TGF-b₁.

Neural Medium: DMEM/F12w/10% FBS, 10% fetal calf serum, 5×10⁻⁷M R-mercaptoethanol (β-2-ME), 10⁻³ M trans retinoic acid and 10% neural basal media (Cambrex).

Smooth Muscle Medium: DMEM/F1 w/10% fetal calf serum.

Fibroblast Medium. DMEM w/10% fetal calf serum, EGF, FGF and 10 ng/ml TGF-b₁.

In addition, the ICC scaffolds can be used to test co-cultures of the ESCs with skin-relevant cell lines including epithelial, endothelial, fibroblast and astrocytes, and observe the differentiation induced by the presence of these cells. When performing co-culture, the ESCs or the co-cultured cells will be stained with calcein or CFSE so that they are distinct from each other under confocal or fluorescence microscopy. The culture will be inspected using an in-house Nikon inverted fluorescence microscope daily to trace the CFSE stained cell, and also be fixed in 2% paraformaldehyde for confocal microscopy analysis. Scanning electron microscopy analysis can be used to assess the cells.

The ESCS will be analyzed for specific markers of cell differentiation for each cell lineage evaluated. Adipogenic development is determined after staining of cells with a dye Oil Red O. Chondrogenic development is evaluated using Safranin O histochemical analysis or immunocytochemical staining for type II collagen. Smooth muscle development is determined after immunohistochemical staining for anti-human alpha-actin. Neuronal development is determined after immunohistochemical staining for anti human nestin, alpha-tubulin and neuron specific nuclear protein.

Skin healing using stem cells and ESCAS is animportant part of the step on this pathway will be the transfer of the differentiation procedures to human skin stem cells (HSSC). HSSCs will be etracted from the burn tissue as well as from the tissues left from cosmetic surgeries (face lifts, tummy tucks, etc) performed in any surgical suite. Skin derived stem cells similarly to ESCs can be much more suitable for the treatment of vesicant injuries because they provide both epidermal as well as dermal components (sweat glands, hair, fat, etc). This can potentially reduce or eliminate the disfiguring scarring occurring in most chemical or thermal burns. It is also envisioned that the use of HSSCs in skin repair can help form a more natural and functional skin tissue with most of the skin's components in place. This differentiates HSSCs from keratinocytes that can be purchased to achieve the same goal. The latter, however, represents only epidermis and these cells are not sufficient for the regeneration of the fully functional skin.

### Example 4

### Effects of Methotrexate and Erythropoietin on Cell Function

Observation of the effect of currently used drugs on the constructed bone marrow replica can be used to validate the entire concept of drug testing ex vivo and will make possible, the development of a standard protocol for the evaluation of specific activity of drug candidates. It will also provide technological foundation for the manufacturing of ex-vivo testing kits for pharmaceutical industry. The drugs, which are known to result in up- and down-regulation of bone marrow in humans, are hypothesized to produce a similar effect in ex-vivo replicas of bone marrow. Two drugs with well characterized effect on the bone marrow including Methotrexate (MT) (also known as Amethopterin, Rheumatrex, Trexall) and erythropoietin (EPO) (also known as Aranesp, Eprex, and NeoRecormon; similar drugs also include CERA and Dynepo). MT is as an antimetabolite drug and can be considered as a representative of a large class of anticancer drugs with similar action against rapidly dividing cells. It is also used in treatment of autoimmune deceases such as psoriasis and rheumatoid arthritis. It is known to inhibit the bone marrow function and, most likely, replication of CD34+ cells. In fact, most of chemotherapy drugs has inhibitory side effect on bone marrow, and therefore, testing with ex-vivo bone marrow can be one of the key tests in drug development.

Another drug/medicament, EPO, is a cytokine for erythrocyte precursors in the bone marrow. It is produced by the kidneys, and is used a therapeutic agent in treating anemia resulting from chronic renal failure or from cancer chemotherapy. It is believed to be common as a blood doping agent in endurance sports. EPO is up-regulating bone marrow function boosting the production of hematopoietic cells and, in particular, HSCs.

Testing of down-regulatory effect of MT on ex-vivo bone marrow. Bone marrow replicas will be subjected to various concentrations of MT. Based on the clinical dosage of MT for adult patients, i.e. 5-15 mg, 0-200 ng of MT per well was used in these experiments. A bone marrow replicas will be incubated to reach a confluent cell layer on the scaffold and after that, will be exposed to 0, 10, 50, 75, 100, 150, 200 ng of MT per well. Each experiment will be repeated 7 times to accumulate sufficient statistical information. The population of the cells in each well will be assessed on Day 1, 2, 3, 4, 5, 7, and 10 after addition of MT. The cells will be analyzed in terms of total cell count, which will provide important information on hematopoietic functions of the bone marrow replica. The population of the cells with the following markers will be assessed: CD34+, CD10, CD19, CD21, CD1a, CD3, CD4, CD8, CD36, CD47, CD71 and IgM. Other cluster of differentiation molecules (CD) will be tested as well. The drop of cell count with CD34+ markers will indicate inhibition of HSC reproduction by MT. CD10 and CD1a will be used to identify B cell and T cell precursors, respectively. CD19, CD21 and IgM will help us to understand the effect of MT on differentiation of CD34+ HSCs into B-cells. CD3, CD4 (helper T-cells), and CD8 (cytotoxic T cells) will distinguish the produced T-cells (if any). The data on the overall cell count and cell count for each marker is to be compared to the blank experiments and correlated with the amount of MT added. The correlation function is established; the threshold of significance will be considered to be that with r value equal or above 0.65 (r=1 is the perfect correlation).

Testing of up-regulatory effect of EPO on ex-vivo bone marrow. Evaluation of effect of EPO on the functionality of the ex-vivo bone replica will follow the same protocol as for MT. Bone marrow replicas obtained as a result of culturing hematopoeitic cells in hydrogel ICC cell scaffolds will be subjected to various amounts of EPO. Each experiment will be repeated 7 times and the population of the cells with different amounts of EPO will be assessed on Day 1, 2, 3, 4, 5, 7, and 10 after addition of EPO. Similar correlations analysis between the concentration of EPO and the total cell output as well as flow cytometry cell counts for CD34+, CD10, CD19, CD21, CD1a, CD3, CD4, CD8, CD36, CD47, CD71, IgM, and other markers can be established. A substantial increase in specific blood cells and overall acceleration of cell proliferation in bone marrow replicas is expected.

### Example 5

### Functional Liver Tissue Constitution for In-Vitro Toxicology Screening of New Drug Compounds.

ICC scaffolds for this purpose provide an ideal 3D microenvironment for reorganization of primary or transformed hepatocytes to form uniform size cell spheroids. The ICC scaffold geometry supports intense cell-to-cell contacts, and hydrogel matrix minimizes cell-to-matrix interactions. As a result, cells seeded on an ICC scaffold form spheroids in a relatively short time, which significantly improves hepatocytes viability and functionality. Furthermore, the spherical shape of pores constrains the size of cell spheroids. Cell spheroids that are fairly uniform in size are formed over the ICC scaffold.

The template of an ICC scaffold is prepared with soda lime glass beads which have diameter less than 200 μg. Highly ordered and packed colloidal crystals are made following the previously mentioned method. Its dimensions exactly fit the size of a single well of the standard 96 well-plate. acrylamide hydrogel precursor solution is infiltrated into the colloidal crystals and polymerized followed by adding an initiator. Glass beads are dissolved by 5% hydrogen fluoride (HF) solution. To completely diffuse away HF from a hydrogel matrix, ICC scaffolds are thoroughly washed with 2 DI-water (PH=2) or PBS buffer solution several times. Then the ICC scaffolds are freeze dried to evaporate remaining HF. Dried ICC scaffolds are rehydrated with PBS buffer solution and sterilized by 70% ethanol followed by 3 hours UV treatment.

HepG2, a transformed human hepatoblastoma cell line, or other human or mouse primary hepatocytes will be used. The media composition will be William's E medium supplemented with 10% FBS, 0.5 μg/ml insulin, 10⁻⁷M dexamethasone, and 1% antibiotics. Approximately 1 million trypsinzed cells will be seeded on one ICC scaffold. To improve cell seeding efficiency, cells seeding will be assisted by centrifugation. An ICC scaffold will be put on a 500 μL capacity centrifugal filter device which has 0.65 μg pore size. One million cells in a 500 μL suspension will be seeded on top of the scaffold, and it will be centrifuged at 1000 rpm for 5 min. Cell seeded ICC scaffolds will be transferred to a 96 well-plate.

The cell spheroids are normally formed within 3 days. On day 1, 3, 5 and 7, the medium and scaffolds samples will be collected. The viability and morphological change of cells on scaffolds will be examined under a confocal microscope utilizing a standard live-dead cell assay kit and scanning electron microscope, respectively. Albumin secretion will be analyzed using an ELISA with purified albumin standard and albumin fluorescence reagent. The cells will be treated with 1 mM NH₄Cl for 4 hours and the produced urea will be measured using an ELISA with dehydrogenase assay kit. At the end of culture, a MTT assay or dsDNA quantification will be performed. These results will be used to normalize ELISA data depending on the actual cell numbers residing in ICC scaffolds.

Once confirmed maintained cell viability and basic functionality of hepatocytes, its ability to produce cytochrome P450 (CYP) will be tested using a training set of chemical compounds. Five distinct inducers such as 3-methylcholanthrene, Phenobarbital, Rifampin, Isoniazid, and Efavirenz, will be added in the hepatocytes model system and released inducer-specific CYP isozymes will be characterized by isozyme identification reagents. Three different concentrations of inducers (10 μM, 5 μM, and 2.5 μM) will be added in culture medium and incubated for 48 h with a replacement of medium at 24 h. The released CYP isozymes will be characterized using an ELISA with identification reagent. After confirming, CYP isozymes secretion potential, biotransformation capability and standardization of the model system activity will be validated by applying a training set of fully characterized CYP inhibitors/inducers in-vivo experiments will be used. The combination of each CYP isozyme specific substrate/inducers or substrate/inhibitors will be added in the hepatocytes model system. Enzyme activities corresponding to the concentration of inducers and inhibitors will be quantitatively characterized by measuring fluorescent intensity. Vivid®CYP substrates will release fluorescent light after consumed by CYP isozymes.

1. A three dimensional inverted colloidal crystal scaffold comprising:
a substrate having at least one well; and
a three dimensional biocompatible polymer matrix comprising a transparent polymer network containing microspherical voids, wherein the microspherical voids are each connected to at least one other void through inter-connecting pores.

2. The three dimensional inverted colloidal crystal scaffold according to claim 1, wherein the polymer matrix comprises a polymer selected from group consisting of polystyrene, collagen gel, fibrin gel, poly(lactic acid), polypeptides, as well as co-polymers of these compounds, hydrogels, bioglasses, inorganic gels and combinations thereof.

3. The three dimensional inverted colloidal crystal scaffold according to claim 2, wherein the hydrogel polymer is selected from the group consisting of poly(acrylamide), poly(acrylates), poly(methacrylates), poly(acrylic acid), poly(urethane), poly(vinyl acetate), collagen, gelatin, alginate, pectin, polyamides, poly(saccharides), and combinations thereof.

4. The three dimensional inverted colloidal crystal scaffold according to claim 1 further comprising a solid substrate having at least one well wherein the three dimensional inverted colloidal crystal scaffold is disposed in the at least one well of the solid substrate.

5. The three dimensional inverted colloidal crystal scaffold according to claim 1, wherein the polymer network comprises an LBL coating.

6. The three dimensional inverted colloidal crystal scaffold according to claim 5, wherein the LBL coating comprises a polyelectrolyte selected from the group consisting of poly(diallydimethyl) ammonium chloride, clay, metal oxides, non-metal oxides, poly-lysine, poly acetylamine, collagen, extracellular matrix, nanocolloidal cellulose, cellulose derivatives, carbon and combinations thereof.

7. The three dimensional hydrogel inverted colloidal crystal scaffold according to claim 6, wherein the polyelectrolyte is poly(diallydimethyl) ammonium chloride and clay.

8. The three dimensional inverted colloidal crystal scaffold according to claim 1, wherein the scaffold further comprises a bioactive agent selected from the group consisting of pharmaceuticals, drugs, toxins, growth factors, differentiation factors, cytokines, antigens, antibodies, differentiation factors, hormones, and combinations thereof.

9. The three dimensional inverted colloidal crystal scaffold according to claim 1, whereon the porous polymeric network formed comprises microspherical voids having an average diameter ranging from about 10 μm to about 500 μm.

10. The three dimensional inverted colloidal crystal scaffold according to claim 1, wherein the microspherical void has at least 6 inter-connecting pores wherein each pore connects to another microspherical void.

11. The three dimensional inverted colloidal crystal scaffold according to claim 1, wherein the diameter of the inter-cavity pore formed within the microspherical void is between about 5 μm and about 25 μm.

12. The three dimensional inverted colloidal crystal scaffold according to claim 1, wherein the polymeric network is transparent when the inverted colloidal crystal scaffold is immersed in a liquid.

13. The three dimensional inverted colloidal crystal scaffold according to claim 1, wherein the scaffold further comprises a living cell.

14. The three dimensional inverted colloidal crystal scaffold according to claim 13, wherein the living cell is a human cell selected from the group consisting of myocytes, fibroblasts, hepatocytes, chondrocytes, osteoblasts, endothelial cells, epithelial cells, stem cells, neural cells, neuronal cells, and combinations thereof.

15. A method of producing an inverted colloidal crystal scaffold, the method comprising:
a) providing a substrate comprising one or more wells;
b) introducing a plurality of microspheres into each well;
c) forming a colloidal crystal template of the plurality of microspheres, the colloidal crystal template comprising a plurality of microspheres and interstitial spaces therebetween;
d) heating the microspheres to partially melt and form junctions with each other;
e) contacting a biocompatible hydrogel polymer precursor around the microspheres;
f) polymerizing the hydrogel polymer precursor to form an integrated three dimensional polymer network; and
g) removing the microspheres in the three dimensional polymer network thereby forming an inverted colloidal crystal scaffold comprising a polymer network with interconnected spherical voids.

16. The method of claim 15, wherein the heating step d) comprises heating the microspheres to a temperature ranging from about 660° C. to about 850° C. to anneal the microspheres together.

17. The method of claim 15, wherein the introducing of microspheres of step b) is achieved by an automated microplate pipetting means comprising a plurality of micropipette tips are arranged in a row above a plurality of wells, wherein the microplate pipetting means delivers accurate volumes of microspheres into the wells.

18. The method of claim 15, wherein step e) further comprises placing the substrate containing the microspheres and the polymer precursor in an ultrasonic bath and agitating the substrate until the polymer precursor has filled a majority of the interstitial spaces between the microspheres.

19. The method of claim 15, wherein the polymerizing step f) comprises polymerizing the polymer precursor using UV radiation, ion beam radiation, and chemical cross-linkers.

20. The method of claim 15, wherein the providing a substrate further comprises providing a substrate with recirculating channels below a membrane supporting the inverted colloidal crystal scaffold.

21. The method of claim 15, wherein the inverted colloidal scaffold is formed in a substrate containing one well having a square shape, and individual inverted colloidal scaffolds are prepared by cutting a plurality of scaffolds from the square shaped substrate.

22. An apparatus for producing a hydrogel inverted crystal scaffold having a polymer network with spherical voids, the apparatus comprising:
a) substrate comprising at least one well;
b) an automated dispensing means for dispensing at least one reagent selected from the group consisting of microspheres, ethylene glycol, water, phosphate buffered saline, polymeric precursor, hydrofluoric acid, and combinations thereof into the at least one well;
c) an agitating apparatus on which the substrate is mounted for agitation therewith;
d) an oven adjacent to the agitating apparatus to remove excess solvent and anneal the microspheres in the substrate by heating the substrate and microspheres to a temperature between 660° C. and 850° C.;
e) a source of actinic radiation adjacent to the oven to polymerize the polymeric precursor applied by the automated dispensing means; and
f) a circulating water bath adjacent to the over in which the dried substrate is immersed to rehydrate the inverted colloidal crystal scaffold and to remove excess hydrofluoric acid.

23. A method of culturing living cells comprising:
providing a cell culture plate having a well and a three dimensional hydrogel inverted colloidal crystal scaffold within the well, wherein the scaffold comprises a biocompatible polymeric network containing microspherical voids, the microspherical voids are each connected to at least one other void through inter-connecting pores;
coating at least a portion of the matrix and at least some of the pores with at least one polyelectrolyte and at least one bioactive agent; and
seeding the living cells into the well and the three dimensional colloidal crystal scaffold.

24. The method according to claim 23, wherein the method further comprises feeding the cells in the scaffold with media for the growth and development of the seeded cells.

25. The method according to claim 23, wherein the seeding of a living cell comprises a physical transfer of the living cell by centrifugation, filtration, spraying and liquid dispensing into the inverted crystal colloidal scaffold.

26. The method according to claim 23 further comprising coating a portion of the matrix and at least some of the pores by coating the scaffold with at least one polyelectrolyte solution and then coating the scaffold with a solution containing the bioactive agent.

27. A method of identifying the effects of a compound on cell function comprising:
a) administering a compound in vitro to an inverted colloidal crystal scaffold seeded with living cells; and
b) determining the affects of the compound on the living cells by measuring, collecting, or recording information on the cells or products produced by the cells.

28. The method according to claim 27, wherein the administering step a) comprises administering a bioactive agent to the cells in the scaffold.

29. The method according to claim 27, wherein the determining step b) further comprises determining changes in cell function that can be measured using techniques comprising Western blot analysis, Northern blot analysis, RT-PCR, immunocytochemical analysis, flow cytometry, immunofluorescence, BrdU labeling, TUNEL assay, assays of enzymatic activity, high throughput and high content analysis.

30. The method according to claim 27, wherein the living cells comprise bone marrow cells, cardiac myocytes, hepatocytes and neural cells.

31. A commercial kit comprising an inverted crystal colloidal scaffold according to claim 1 sealed in a sterile package and instructions for use thereof for culturing cells.
